(19) United States
(12) Patent Application Publication
(10) Pub. No.: US 2007/0123548 A1
(43) Pub. Date: May 31, 2007

(54) Pth agonists

(76) Inventors: David John Cowan, Research Triangle Park, NC (US); Frank Navas III, Research Triangle Park, NC (US); Jeffrey Alan Oplinger, Research Triangle Park, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/597,858

(22) PCT Filed: Feb. 3, 2005
(86) PCT No.: PCT/US05/03248

§ 371 Date: Aug. 10, 2006

Related U.S. Application Data
(60) Provisional application No. 60/543,517, filed on Feb. 11, 2004.

(51) Int. Cl. A61K 31/515; C07D 239/60

(52) U.S. Cl. 514/269; 514/270; 544/301; 544/311

(57) ABSTRACT

This invention relates to compounds of formula (I) or (II) 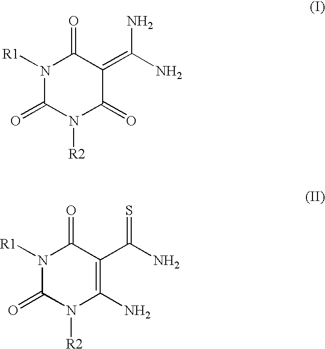 wherein, in either formula, independently;
R¹ and R² are the same or are different and are C₁₋₈ alkyl, C₂₋₈ alkylene, C₃₋₈ cycloalkyl, aryl, heteroaryl, heterocycloalkyl, C₃₋₆ cycloalkylaryl, or heterocycloaryl; wherein said alkyl, alkylene, cycloalkyl, aryl, heteroaryl, heterocyclyl, cycloalkylaryl, or heterocycloaryl are unsubstituted or substituted by one or more groups selected from the group consisting of halogen, C₁₋₈ alkyl, C₁₋₈alkoxy, C₁₋₈thioalkoxy, cycloalkyl, aryl, heteroaryl, heterocycloalkyl, CF₃, SCF₃, NHC(O)ₙR⁵, S(O)ₘR⁵, S(O)₂NR⁵R⁶, C(S)NR⁵R⁶, CONR⁵R⁶, C(O)ₙR⁵; n is 0, 1 or 2; m is 0, 1 or 2;
R⁵ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃;
R⁶ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃, or when R¹ and/or R² contains S(O)₂NR⁵R⁶, CONR⁵R⁶, or C(S)NR⁵R⁶, then R⁵R⁶ together with the nitrogen may form a heterocyclic ring; or a pharmaceutically acceptable salt or solvate thereof.

### SCOPE OF THE INVENTION

This invention relates to uracil-derived compounds that are agonists of the parathyroid hormone type I receptor (PTH1R) and as such is useful for the treatment of osteoporosis.

### BACKGROUND OF THE INVENTION

Osteoporosis is characterized by bone loss resulting in an increased incidence of fracture. This condition, which is most prevalent in the spine and hip, affects 1 in 3 postmenopausal women, a lesser but significant number of aging men, and is also caused by other conditions including hypogonadism and prolonged glucocorticoid use.

All current therapies to treat osteoporosis, such as bisphosphonates, hormone replacement therapy, SERMs and calcitonin, serve to arrest further bone loss by inhibiting bone resorption (Sato M, et al; 1999, J. Med. Chem. 42:1-24). However, although continued bone loss may be slowed or even prevented by these treatments, new bone formation leading to increased bone mass and strength, does not occur. Consequently, there is a considerable demand for a therapeutic agent capable of stimulating bone formation and that could be used either alone or in combination with an anti-resorptive agent to reduce further risk of fracture. Such a therapeutic agent would be beneficial both to patients who are at risk of developing osteoporosis or who present with established osteoporosis.

Parathyroid hormone (PTH) is a major regulator of calcium homeostasis and acts, in part, by mobilizing calcium from the skeleton through increased bone resorption. Additionally, pulsatile administration of PTH has repeatedly been demonstrated to stimulate new bone formation, both in laboratory animals and in humans (Hock J M, Gera I. 1992. J. Bone Miner. Res. 7:65-72; Wronski T J, et al, 1993, Endocrinology 132:823-831; and Reeve J, et al, 1980, Br Med J. 280:134-1344). As such, it is the only agent known to stimulate bone formation on previously quiescent bone surfaces (Hodsman A B, et al, Bone 14:523-527 and Dobnig H, Turner R T. 1995, Endocrinology 136:3632-3638). Indeed, hPTH(1-34), an N-terminal fragment of human PTH that appears to exhibit equivalent bone anabolic activity to the full-length hormone [PTH(1-84)], has been developed by Eli Lilly for the treatment of osteoporosis (Forteo/Teriparatide), as has recombinant human PTH(1-84) by Allelix (Ashworth L E, 2002, Formulary 37:129-139). A recombinant human parathyroid hormone fragment with anabolic actions for treatment of osteoporosis. Formulary 37:129-139) In a clinical trial, PTH(1-34) administered by daily subcutaneous injection for up to 2 years to postmenopausal women with prior vertebral fractures, was reported to reduce fracture incidence at the spine and non-vertebral sites by 65 and 40%, respectively (Neer R M, et al, N. Engl. J. Med. 344:1434-1441).

Taken together, there is overwhelming evidence to suggest that targeting of the receptor for PTH with a small molecule agonist mimicking the actions of PTH(1-34), would be a suitable approach for generating an anabolic response in bone.

PTH elicits its effects by binding and activating a class B G protein-coupled receptor of the 7 transmembrane superfamily, designated PTH1R (Abou-Samra A-B, et al, Proc. Natl. Acad. Sci. USA 89:2732-2736). The PTH1R activates multiple signaling pathways, but predominantly the adenylyl cyclase/cyclic AMP and the phospholipase C/calcium mobilization pathways. Evidence from the literature suggests that activation of the cAMP pathway is necessary but not sufficient for the bone anabolic response (Hock J M, et al, Endocrinology 125:2022-2027 and Rixon R H, et al, J Bone Miner. Res. 9:1179-1189). Both these responses were utilized to identify PTH1R activators (agonists) in screening compounds for agonist activity.

The goal of this invention is to provide a small molecule that mimicks the desired bone anabolic effects of PTH through targeting of the PTH1R, but which can be administered orally rather than by injection. This would offer significant benefits both in terms of lower production costs versus a peptide as well as ease of administration to the patient. Such compounds are provided herein.

### SUMMARY OF THE INVENTION

In one aspect, this invention relates to compounds of formula (I) or (II) 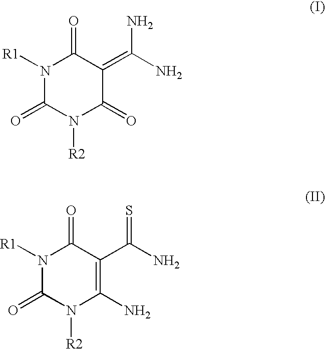
wherein, in either formula, independently;

R¹ and R² are the same or are different and are C₁₋₈ alkyl, C₂₋₈ alkylene, C₃₋₈ cycloalkyl, aryl, heteroaryl, heterocycloalkyl, C₃₋₆cycloalkylaryl, or heterocycloaryl; wherein said alkyl, alkylene, cycloalkyl, aryl, heteroaryl, heterocyclyl, cycloalkylaryl, or heterocycloaryl are unsubstituted or substituted by one or more groups selected from the group consisting of halogen, C₁₋₈ alkyl, C₁₋₈alkoxy, C₁₋₈thioalkoxy, cycloalkyl, aryl, heteroaryl, heterocycloalkyl, CF₃, SCF₃, NHC(O)ₙR⁵, S(O)ₘR⁵, S(O)₂NR⁵R⁶, C(S)NR⁵R⁶, CONR⁵R⁶, C(O)ₙR⁵;

n is 0, 1 or 2;

m is 0, 1 or 2;

R⁵ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃;

R⁶ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃, or when R¹ and/or R² contains S(O)₂NR⁵R⁶, CONR⁵R⁶, or C(S)NR⁵R⁶, then R⁵R⁶ together with the nitrogen may form a heterocyclic ring; or

a pharmaceutically acceptable salt or solvate thereof.

In another aspect, the present invention includes pharmaceutical compositions comprising a compound of formula (I) and/or (II), or a salt or solvate thereof in admixture with a pharmaceutically acceptable excipient, or mixtures thereof.

Another aspect of this invention is a means for preventing or treating a condition mediated by PTH which comprises administering to a mammal in need thereof an effective amount of a compound of formula (I) or (II), salts or solvates thereof, or mixtures thereof either alone or in admixture with a pharmaceutically excipient.

Another aspect of the invention includes compounds of formula (I) or (II), or mixtures thereof for use in the treatment and prevention of diseases and conditions characterised by loss of bone mineral density, mass, or strength, as well as in conditions wherein PTH would have a beneficial pharmacological effect.

Another aspect of the invention includes administering compounds of formula (I) or (II) for use as a PTH mimetic.

Another aspect of the invention includes use of the compounds of formula (I) or (II) or mixtures thereof in the manufacture of a medicament for use in the treatment of osteopenia and osteoporosis in men and women for reduction in the risk of fractures, both vertebral and nonvertebral.

### DETAILED DESCRIPTION OF THE INVENTION

As used herein, the term “C₁₋₈ alkyl” or “lower alkyl” refers to an alkyl group containing at least 1 and at most 8 carbon atoms. Examples of branched or straight-chain “C₁₋₈ alkyl” groups include, but are not limited to methyl, ethyl, n-propyl, isopropyl, isobutyl, n-butyl, and t-butyl, isobutyl, n-pentyl, n-hexyl, n-heptyl, n-octyl and the like.

The term “alkylene” refers to a straight or branched chain unsaturated aliphatic hydrocarbon radical of 2 to 6 carbon atoms that may be optionally substituted, with multiple degrees of substitution being allowed. Examples of “alkylene” include, but are not limited to methylene, ethylene, n-propylene, n-butylene, and the like.

The term “halogen” refers to fluorine, chlorine, bromine, or iodine.

The term “cycloalkyl” refers to an optionally substituted non-aromatic cyclic hydrocarbon ring of 3 to 8 carbons. Exemplary “cycloalkyl” groups include, but are not limited to cyclopropyl, cyclobutyl, cyclopentyl, cyclohexyl, cycloheptyl and cyclooctyl.

The term “heterocycloalkyl” refers to a heterocyclic ring containing one or more heteroatomic substitutions replacing one or more carbons, selected from S, S(O), S(O)₂, O, or N, that may be further optionally substituted, with multiple degrees of substitution being allowed. Such a ring may be optionally fused to one or more other “heterocycloalkyl” ring(s) or cycloalkyl ring(s). Examples of “heterocyclic” moieties include, but are not limited to tetrahydrofuran, pyran, 1,4-dioxane, 1,3-dioxane, piperidine, pyrrolidine, morpholine, tetrahydrothiopyran, tetrahydrothiophene, and the like.

The term “aryl” refers to a benzene ring or to an optionally substituted benzene ring system fused to one or more optionally substituted benzene rings to form, for example, anthracene, phenanthrene, or naphthalene ring systems. Examples of “aryl” groups include, but are not limited to phenyl, 2-naphthyl, 1-naphthyl, biphenyl, as well as substituted derivatives thereof. The term “lower alkylaryl” further refers to groups of —RₐR_{b}, where Rₐ is a “lower alkyl” as defined herein and R_{b} is an aryl as defined herein.

“Heteroaryl” refers to a monocyclic aromatic ring system, or to a fused bicyclic aromatic ring system comprising two aromatic rings. These heteroaryl rings contain one or more nitrogen, sulfur, and/or oxygen atoms, where N-oxides and sulfur oxides and dioxides are permissible heteroatom substitutions and may be optionally substituted, with multiple degrees of substitution being allowed. Examples of “heteroaryl” groups used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof. The term “lower alkylheteroaryl” further refers to groups of —RₐR_{b}, where Rₐ is a “lower alkyl” group as defined herein and R_{b} is a heteroaryl as defined herein.

“Alkoxy” refers to the group RₐO—, where Rₐ is alkyl or aryl as defined above. The term “thioalkoxy” refers to the group RₐS—, where Rₐ is alkyl or aryl as defined above. The term “alkoxyaryl” refers to the group R_{b}RₐO—, where Rₐ is alkyl and R_{b} is aryl as defined above.

The terms “C₃₋₆cycloalkylaryl” and “heterocyclylaryl” means a group of —RₐR_{b} where Rₐ is a cycloalkyl or heterocycloalkyl respectively that is fused with R_{b} which is defined as an aryl group. Examples of such groups include:

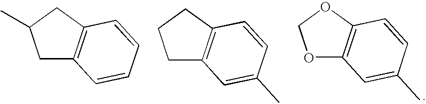

Preferably R¹, R² are the same or are different and are independently C₃₋₆ alkyl, C₃₋₆ alkylene, C₃₋₈cycloalkyl, C₄₋₆ alkylaryl, C₃₋₄cycloalkylaryl, heterocycloaryl or heterocycloalkyl. Said C₃₋₆ alkyl or heterocycloalkyl may be optionally substituted with NHC(O)ₙR⁵ or C(O)ₙR⁵ wherein n is 2 and R⁵ is lower alkylaryl as herein defined wherein said lower alkylaryl may be optionally substituted with one or more groups selected from F, NO₂, or N₃.

More preferably, R² is n-butyl and R¹ is

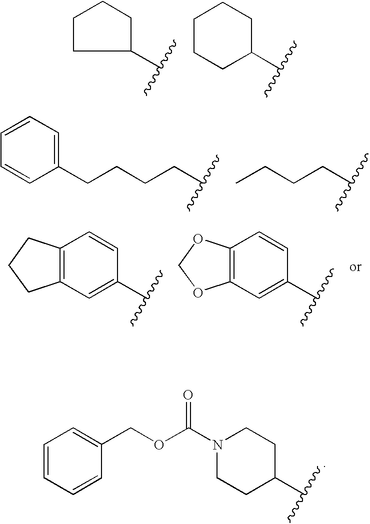

Preferred compounds of formula (I) include:
1,3-dicyclohexyl-5-(diaminomethylene)pyrimidine-2,4,6(1H,3H,5H)-trione,
1-butyl-5-(diaminomethylene)-3-(2-methylbutyl)pyrimidine-2,4,6(1H,3H,5H)-trione,
1-butyl-5-(diaminomethylene)-3-(2,3-dihydro-1H-inden-2-yl)pyrimidine-2,4,6(1H,3H,5H)-trione,
1-butyl-5-(diaminomethylene)-3-{4-[(trifluoromethyl)thio]phenyl}pyrimidine-2,4,6(1H,3H,5H)-trione,
1-butyl-5-(diaminomethylene)-3-mesitylpyrimidine-2,4,6(1H,3H,5H)-trione,
1-butyl-5-(diaminomethylene)-3-(2,4-difluorophenyl)pyrimidine-2,4,6(1H,3H,5H)-trione,
1-butyl-5-(diaminomethylene)-3-(2-fluorophenyl)pyrimidine-2,4,6(1H,3H,5H)-trione,
1-butyl-3-(cyclohexylmethyl)-5-(diaminomethylene)pyrimidine-2,4,6(1H,3H,5H)-trione,
1-butyl-3-cycloheptyl-5-(diaminomethylene)pyrimidine-2,4,6(1H,3H,5H)-trione,
1-butyl-3-cyclooctyl-5-(diaminomethylene)pyrimidine-2,4,6(1H,3H,5H)-trione,
1-butyl-5-(diaminomethylene)-3-(3-phenylcyclopentyl)pyrimidine-2,4,6(1H,3H,5H)-trione,
1-butyl-5-(diaminomethylene)-3-(5-phenylpentyl)pyrimidine-2,4,6(1H,3H,5H)-trione,
1-[3-(benzyloxy)phenyl]-3-butyl-5-(diaminomethylene)pyrimidine-2,4,6(1H,3H,5H)-trione,
benzyl 3-[3-butyl-5-(diaminomethylene)-2,4,6-trioxotetrahydropyrimidin-1(2H)-yl]propylcarbamate,
4-nitrobenzyl 3-[3-butyl-5-(diaminomethylene)-2,4,6-trioxotetrahydropyrimidin-1(2H)-yl]propylcarbamate,
4-fluorobenzyl 3-[3-butyl-5-(diaminomethylene)-2,4,6-trioxotetrahydropyrimidin-1(2H)-yl]propylcarbamate,
4-(2λ⁵-triaza-1,2-dienyl)benzyl 3-[3-butyl-5-(diaminomethylene)-2,4,6-trioxotetrahydropyrimidin-1(2H)-yl]propylcarbamate,
1-but-3-enyl-3-cyclopentyl-5-(diaminomethylene)pyrimidine-2,4,6(1H,3H,5H)-trione,
4-(2λ⁵-triaza-1,2-dienyl)benzyl 4-[3-butyl-5-(diaminomethylene)-2,4,6-trioxotetrahydropyrimidin-1(2H)-yl]piperidine-1-carboxylate,
benzyl 3-[3-butyl-5-(diaminomethylene)-2,4,6-trioxotetrahydropyrimidin-1(2H)-yl]pyrrolidine-1-carboxylate,
1-butyl-5-(diaminomethylene)-3-(3,5-dimethylisoxazol-4-yl)pyrimidine-2,4,6(1H,3H,5H)-trione,
1,3-dibutyl-5-(diaminomethylene)pyrimidine-2,4,6(1H,3H,5H)-trione,
1-butyl-5-(diaminomethylene)-3-(4-phenylbutyl)pyrimidine-2,4,6(1H,3H,5H)-trione,
benzyl 4-[3-butyl-5-(diaminomethylene)-2,4,6-trioxotetrahydropyrimidin-1(2H)-yl]piperidine-1-carboxylate,
1-butyl-3-cyclopentyl-5-(diaminomethylene)pyrimidine-2,4,6(1H,3H,5H)-trione,
1-butyl-5-(diaminomethylene)-3-(2,3-dihydro-1H-inden-5-yl)pyrimidine-2,4,6(1H,3H,5H)-trione,
1-(1,3-benzodioxol-5-yl)-3-butyl-5-(diaminomethylene)pyrimidine-2,4,6(1H,3H,5H)-trione, and
1-butyl-3-cyclohexyl-5-(diaminomethylene)pyrimidine-2,4,6(1H,3H,5H)-trione.

A preferred compound of formula II is:
6-amino-1,3-dibutyl-2,4-dioxo-1,2,3,4-tetrahydropyrimidine-5-carbothioamide.

Certain of the compounds described herein contain one or more chiral atoms, or may otherwise be capable of existing in enantiomeric and diastereomeric forms. The scope of the present invention is intended to cover all isomers per se, as well as mixtures of cis and trans isomers, mixtures of diastereomers, and racemic mixtures of enantiomers. Also included within the scope of the invention are the individual isomers of the compounds represented by formulas (I) or (II) above as well as any wholly or partially equilibrated mixtures thereof. The present invention also covers the individual isomers of the compounds represented by the formulas above as mixtures with isomers thereof in which one or more chiral centers are inverted.

As noted above, the present invention includes salts and solvates of the compounds of the present invention. Salts include addition salts, metal salts, or optionally alkylated ammonium salts. Examples of such salts include hydrochloric, hydrobromic, hydroiodic, phosphoric, sulfuric, trifluoroacetic, trichloroacetic, oxalic, maleic, pyruvic, malonic, succinic, citric, mandelic, benzoic, cinnamic, methane sulphonic, ethane sulphonic, picric, and the like. Further salts include lithium, sodium, potassium, magnesium, and the like. Reference is also made to Journal of Pharmaceutical Science, 1997, 66, 2, incorporated herein by reference as relevant to salts.

As used herein, the term “solvate” refers to a complex of variable stoichiometry formed by a solute (in this invention, a compound of formula (I) or (II) or a salt or physiologically functional derivative thereof) and a solvent. Such solvents for the purpose of the invention should not interfere with the biological activity of the solute. Examples of solvents include, but are not limited to water, methanol, ethanol, and acetic acid. Preferably the solvent used is a pharmaceutically acceptable solvent. Examples of pharmaceutically acceptable solvents include water, ethanol, and acetic acid.

While it is possible that compounds of the present invention may be administered as the raw chemical, preferably the compounds of the present invention are presented as an active ingredient within a pharmaceutical formulation, as are known in the art. Accordingly, the present invention further includes a pharmaceutical formulation comprising a compound of formula (I) or (II), or salt, solvate, or functional derivative thereof together with one or more pharmaceutically acceptable carriers. Optionally, other therapeutic and/or prophylactic ingredients may be included in the pharmaceutical formulation. For example, the compounds of the present invention may be combined with other agents useful in the treatment or prophylaxis of osteoporosis, such as calcium, PTH, Vitamin D, estrogen, SERMs, bisphosphonates, and the like.

Formulations of the present invention include those especially formulated for oral, buccal, parental, transdermal, inhalation, intranasal, transmucosal, implant, or rectal administration. Among the variety of administrations, oral administration typically is preferred. For oral administration tablets, capsules, and caplets may contain conventional excipients such as binding agents, fillers, lubricants, disintegrants, and/or wetting agents. Non-limiting examples of binding agents include syrup, acacia, gelatin, sorbitol, tragacanth, mucilage of starch, or polyvinylpyrrolidone (PVP). Non-limiting examples of fillers include, for example, lactose, sugar, microcrystalline cellulose, maize-starch, calcium phosphate or sorbitol. Non-limiting examples of lubricants include, for example, magnesium sterate, stearic acid, talc, polyethylene glycol or silica. Non-limiting examples of disintegrants include, for example, potato starch or sodium starch glycollate. A non-limiting example of a wetting agent includes sodium lauryl sulfate. The tablets additionally may be coated according to methods known in the art.

Alternatively, the compounds of the present invention may be incorporated into oral liquid preparations such as aqueous or oily suspensions, solutions, emulsions, syrups, or elixirs. Moreover, formulations containing these compounds may be presented as a dry product for constitution with water or other suitable vehicle before use. Liquid preparations may contain conventional additives. Non-limiting examples of such additives include suspending agents such as sorbitol syrup, methyl cellulose, glucose/sugar syrup, gelatin, hydroxyethylcellulose, carboxymethyl cellulose, aluminum sterate gel or hydrogenated edible fats. Additionally, emulsifying agents such as lecithin, sorbitan mono-oleate or acacia; non-aqueous vehicles (which may include edible oils) such as almond oil, fractionated coconut oil, oily esters, propylene glycol or ethyl alcohol my be included. Further, preservatives such as methyl or propyl p-hydroxybenzoates or sorbic acid, may be incorporated into the preparation. Such preparations may also be formulated as suppositories, for example, containing conventional suppository bases such as cocoa butter or other glycerides.

Additionally, formulations of the present invention may be formulated for parenteral administration by injection or continuous infusion. Formulations for injection may take such forms as suspensions, solutions, or emulsions in oily or aqueous vehicles, and may contain formulatory agents such as suspending, stabilising and/or dispersing agents. Alternatively, the active ingredient may be in powder form for constitution with a suitable vehicle, for example, sterile, pyrogen-free water, before use.

The formulations according to the invention may also be formulated as a depot preparation. Such long acting formulations may be administered by implantation, for example, subcutaneously or intramuscularly, or by intramuscular injection. Accordingly, the compounds of the invention may be formulated with suitable polymeric or hydrophobic materials, such as an emulsion in an acceptable oil, ion exchange resins, or as sparingly soluble derivatives, such as a sparingly soluble salt.

Pharmaceutical formulations may be presented in unit dose forms containing a predetermined amount of active ingredient per unit dose. Such a unit may contain certain amounts of a compound of formula (I) and/or (II) depending on the condition being treated, the route of administration, and the age, weight and condition of the patient. Preferred unit dosage formulations are those containing a predetermined dose, such as a daily dose, or an appropriate fraction thereof, of an active ingredient. Such pharmaceutical formulations may be prepared by any of the methods well known in the pharmacy art.

A “therapeutically effective amount” of a compound of the present invention will depend upon a number of factors including, for example, the age and weight of the animal, the precise condition requiring treatment and its severity, the nature of the formulation, and the route of administration. Therapeutic effectiveness ultimately will be at the discretion of the attendant physician or veterinarian. An effective amount of a salt or solvate, or physiologically functional derivative thereof, may be determined as a proportion of the effective amount of the compound of formula (I) and/or (II) per se.

No unacceptable toxicological effects are expected when compounds of the present invention are administered in accordance with the present invention.

### METHODS OF PREPARATION—DETAILED DESCRIPTION

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.
Preparation of Compounds of Formula I 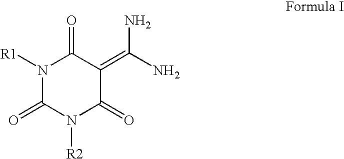
may be prepared from compounds of Formula IV 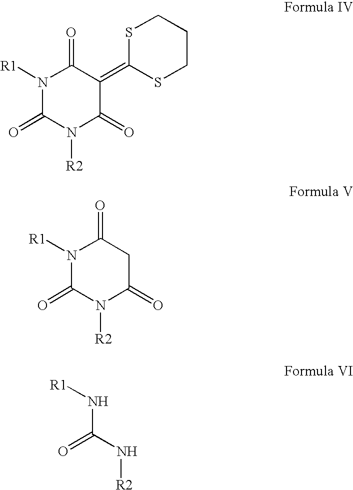
in a polar, protic solvent such as methanol in the presence of ammonia at temperatures from 20° C. to 150° C., such as refluxing methanol. Compounds of Formula IV may be prepared from compounds of Formula V in a polar solvent, such as dimethylsulfoxide, by treating compounds of Formula V with a base, such as triethylamine in the presence of carbon disulphide at temperatures of from 0° C. to 100° C., such as 23° C., and treating such mixtures with 1,3-dibromopropane at temperatures of from 0° C. to 100° C., such as 23° C. Compounds of Formula V may be prepared from compounds of formula VI in a polar protic solvent, such as acetic acid, in the presence of acetic anhydride and 1 equivalent of malonic acid at temperatures of from 20° C. to 150° C., such as 80° C. for 2 hrs. Compounds of Formula VI are commercially available or may be easily prepared by one skilled in the art.
Preparation of Compounds of Formula II 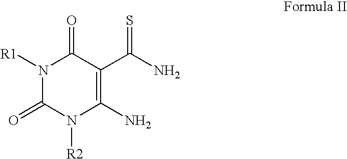
may be prepared from compounds of Formula VII 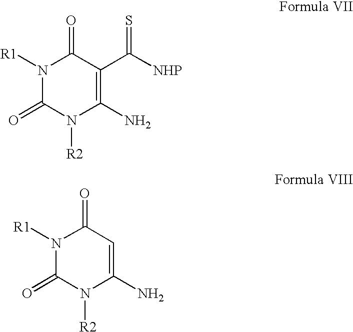
by deprotection of the nitrogen protecting group. Such a protecting group is the 4-methoxy-benzyl protecting group, removed under acidic conditions, such as HBr in acetic acid at temperatures from 20-150° C., such as 80° C. Compounds of formula VII may be prepared from compounds of formula VIII in a polar aprotic solvent, such as DMF, at temperatures from 20-150° C., such as 100° C. in the presence of a suitable isothiocyanate. The isothiocyanates are commercially available or may be readily prepared by one skilled in the art. Compounds of formula VIII may be prepared from compounds of formula VI by one skilled in the art (see J. Med. Chem. 1994, 37(20) 3373-3382).

This invention further provides a method for treating osteoporosis or inhibiting bone loss which comprises internal administration to a patient of an effective amount of a compound of Formula I, alone or in combination with other inhibitors of bone resorption, such as bisphosphonates (i.e., allendronate), hormone replacement therapy, anti-estrogens, or calcitonin. In addition, treatment with a compound of this invention and an anabolic agent, such as bone morphogenic protein, iproflavone, may be used to prevent bone loss or to increase bone mass.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin K. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

Biological Assay

Compounds of the invention were determined to be agonists of PTH1R using a tagged human PTH1R expressed in CHO cells (Affymax Research, 4001 Miranda Avenue, Palo Alto, Calif. 94304, US) transfected with a cAMP response element (CRE) reporter (EC₅₀=5.3 μM, 73% PTH maximal response). No responses were elicited by compounds of formula (I) in mock-transfected cells, indicating that their stimulatory effects on the cAMP and intracellular calcium ion concentration were PTH1R mediated.

Compounds of formula (I) were found to mimic the effect of PTH(1-34) when added over the concentration range 1-10 μM (EC₅₀ typically ˜1-3 μM), when used in the following assays:

(i) Activation of the cAMP response element-luciferase (CRE-Luc) reporter in HEK cell line expressing human PTH1R (but no response in HEK cells lacking the PTH1R).

(ii) FLIPR/mobilization of intracellular calcium in HEK cells expressing PTH1R.

(iii) Stimulation of cAMP synthesis in the following cells: HEK cells engineered to express the PTH1R; rat osteosarcoma cells (ROS 17/2.8) that express endogenous PTH1R; primary rat osteoblasts isolated from fetal calvariae.

(iv). Stimulation of osteocalcin release from ROS 17/2.8 cells.

(v). Induction of a downstream target gene, RGS-2, in ROS 17/2.8 cells.

(vi). Inhibition of adipocyte differentiation in cultures of human bone marrow stromal fibroblasts when administered in a pulsatile fashion.

(vii). Induce a dose and time-dependent increase blood ionized calcium concentration after single dosing to thyro-parathyroidectomized rat.

Importantly, compounds of formula (I) that were found to be active in the above assays, also caused partial displacement of radio-iodinated (¹²⁵I) (Nle^{8,18})(Tyr³⁴)-PTH(1-34) binding to PTH1R in membrane preparations of HEK cells expressing PTH1R. For these compounds the IC₅₀ value for binding was 2-3 μM, directly coinciding with the concentration range required to observe biological activity.

### EXAMPLES

In the following synthetic examples, temperature is in degrees Centigrade (° C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### 1,3-Dibutyl-5-(diaminomethylene)pyrimidine-2,4,6(1H,3H,5H)-trione

### 1(a) Preparation of intermediate 1,3-dibutylpyrimidine-2,4,6(1H,3H,5H)-trione

The synthesis of barbituric acid compounds has been described (Biltz, H., Wittek, H. Ber. 1921, 54B, 1035-58; Carnell, A. J. et. al. Tetrahedron Lett. 1999, 40, 8029-8032). Thus, to a mixture of 30 g (0.174 mol) N,N′-dibutylurea and 18.2 g (0.174 mol) malonic acid in 200 mL of Acetic acid was added 62.2 g (0.61 mol) of acetic anhydride. The mixture was heated to 80-90° C. with stirring for 2.5 h. The resulting solution was concentrated under vacuum to a brown oil which was flushed through a short plug of silica gel (1 kg) eluting with 5%-50% EtOAc in hexanes to give a yellow colored oil (38.8 g, 93%). Upon standing the oil solidified to the pale yellow solid intermediate 1,3-dibutylpyrimidine-2,4,6(1H,3H,5H)-trione of sufficient purity to carry forward into the next reaction.

### 1(b) Preparation of intermediate 1,3-dibutyl-5-(1,3-dithian-2-ylidene)pyrimidine-2,4,6(1H,3H,5H)-trione

To a solution of 38.3 g (0.159 mol) of intermediate 1,3-dibutylpyrimidine-2,4,6(1H,3H,5H)-trione in 200 mL of DMSO at 22° C. was added 36.4 g (0.478 mol) of carbon disulfide followed by the dropwise addition of 48.4 mL (0.478 mol) of Et₃N. After 1 h, 32.3 g (0.16 mol) of 1,3-dibromopropane was added and stirring was continued at 23° C. for 12 h. The darkened solution was diluted with 500 mL EtOAc and 500 mL of water. The aqueous phase was extracted once with EtOAc and the combined organics were dried over Na₂SO₄, filtered, and concentrated. The resulting crude oil was flushed through a short pad of silica gel eluting with EtOAc in hexanes. After concentrating, the crude oil was taken into 200 mL of boiling 20% EtOAc in hexanes solution. Upon standing at 0° C. for 1 h, the precipitated solids were isolated by filtration and dried to yield 47.4 grams of yellowed solid (83% yield) intermediate 1,3-dibutyl-5-(1,3-dithian-2-ylidene)pyrimidine-2,4,6(1H,3H,5H)-trione.

### 1(c) Preparation of 1,3-dibutyl-5-(diaminomethylene)pyrimidine-2,4,6(1H,3H,5H)-trione

10 g (28 mmol) of 1,3-dibutyl-5-(1,3-dithian-2-ylidene)pyrimidine-2,4,6(1H,3H,5H)-trione was divided equally into 5 Ace Glass pressure tubes and each tube charged with 20 mL (40 mmol) of 2.0 M ammonia in methanol solution. The pressure tubes were sealed, then stirred at ˜100° C. for 2.5 h with the mixtures becoming homogenous after several minutes. Upon cooling, the combined reaction solutions were partially concentrated, then taken up into 3-4 volumes of boiling EtOAc in hexanes. After cooling to 0° C. for 60 min, the white solids were isolated via filtration and dried under vacuum at 60° C. to yield 6.6 g (83.5%) of the title compound: ¹H NMR (300 MHz, CDCl₃) δ 10.2 (br s, 2H), 5.57 (br s, 2H), 3.91 (t, 4H, J=7.6), 1.6 (m, 4H), 1.38 (m, 4H), 0.95 (t, 6H, J=7.4); MS (m/z) 283.17 (MH+, 100%).

### Example 2

### 1,3-Dicyclohexyl-5-(diaminomethylene)pyrimidine-2,4,6(1H,3H,5H)-trione

Available from commercial sources (Zelinsky Institute of Organic Chemistry of the Russian Academy of Sciences (ZIOC).

### Example 3

### 1-Butyl-5-(diaminomethylene)-3-(2-methylbutyl)pyrimidine-2,4,6(1H,3H,5H)-trione

### 3(a) Preparation of intermediate 1-butyl-5-(1,3-dithian-2-ylidene)-3-(2-methylbutyl)pyrimidine-2,4,6(1H,3H,5H)-trione

To a solution of 2-methylbutan-1-amine (1 mmol) in 1.5 mL of dichloromethane at 23° C. was added 1.0 mmol (0.5 mL of 2.0 M dichloromethane solution) of butylisocyanate. The solution was stirred overnight at 23° C., concentrated, and the residues diluted into 1 mL of AcOH and 0.7 mL of water. Malonic acid (1 mmol, 105 mg) was added and the mixture was heated for 5 hrs at 80° C. with stirring. The mixture was then concentrated under reduced pressure at 40° C. over 15 hr and the crude barbituric acid intermediate was taken into 1.5 mL of dimethylsulfoxide before triethylamine (0.56 mL, 4 mmol) and carbon disulfide (0.18 mL, 3 mmol) were added. The mixture was stirred at 23° C. for 1 hr followed by the addition of 0.11 mL of neat 1,3-dibromopropane. After stirring for 9 hr at 23° C., the mixture was diluted with ethyl acetate (10 mL) and water (3 mL) and the crude intermediate product was isolated from the organic phase as an oil. The crude product was purified on a 10 g silica gel column eluting with hexanes/ethyl acetate to yield 280 mg (76% yield) of intermediate 1,3-dibutyl-5-(1,3-dithian-2-ylidene)pyrimidine-2,4,6(1H,3H,5H)-trione.

### 3(b) Preparation of 1-butyl-5-(diaminomethylene)-3-(2-methylbutyl)pyrimidine-2,4,6(1H,3H,5H)-trione

Into a sealable pressure tube was prepared a suspension of 260 mg of intermediate 1,3-dibutyl-5-(1,3-dithian-2-ylidene)pyrimidine-2,4,6(1H,3H,5H)-trione in 5 mL of 2.0 M NH3 in methanol solution. The tube was sealed and the contents stirred at 110° C. for 4 hr. The solution was cooled, partially concentrated (˜2 mL) and set overnight at ˜5° C. The solids were collected by filtration, washed with cold hexanes, and dried under vacuum for several hours to yield 200 mg (67% yield) of the title compound as a white solid: ¹H NMR (300 MHz, CDCl₃) δ 10.4 (br s, 2H), 5.3 (br s, 2H), 3.92 (t, 2H, J=7.6), 3.8 (m, 2H), 1.9 (m, 1H), 1.66-1.41 (series of multiplets, 4H), 1.21 (m, 1H), 0.96 (t, 3H, J=7.4), 0.93 (t, 3H, J=7.5), 0.89 (d, 3H, J=6.8); MS (m/z) 297.3 (MH+, 100%).

### Example 4

### 1-Butyl-5-(diaminomethylene)-3-(2,3-dihydro-1H-inden-2-yl)pyrimidine-2,4,6(1H,3H,5H)-trione

The title compound was prepared identical to that described for the preparation of example 3. Thus, 1 mmol of 2,3-dihydro-1H-inden-2-ylamine gave 120 mg (29% yield) of intermediate 1-butyl-3-(2,3-dihydro-1H-inden-2-yl)-5-(1,3-dithian-2-ylidene)pyrimidine-2,4,6(1H,3H,5H)-trione after chromatography as described. From 110 mg of this dithiane intermediate was obtained 70 mg of the title compound as a white solid: ¹H NMR (300 MHz, CDCl₃) δ 10.3 (br s, 2H), 7.24-7.16 (m, 4H), 5.9 (m, 1H), 5.15 (br s, 2H), 3.91 (t, 2H, J=7.6), 3.59 (dd, 2H, J=15.5, 8.2), 3.23-3.13 (m, 2H), 1.67-1.56 (m, 2H), 1.44-1.31 (m, 2H), 0.9 (t, 3H, J=); MS (m/z) 343.7 (MH+, 100%).

### Example 5

### 1-Butyl-5-(diaminomethylene)-3-{4-[(trifluoromethyl)thio]phenyl}pyrimidine-2,4,6(1H,3H,5H)-trione

### Preparation of dithiane intermediate 1-butyl-5-(1,3-dithian-2-ylidene)-3-{4-[(trifluoromethyl)thio]phenyl}pyrimidine-2,4,6(1H,3H,5H)-trione

To a solution of 2 mmol (420 mg) of 1-isocyanato-4-[(trifluoromethyl)thio]benzene in 1.5 mL of dichloromethane at 23° C. was added 200 uL of n-butylamine. The mixture was stirred overnight at 23° C., filtered free of solids, and poured into ethyl acetate and washed with water and CuSO₄ solutions. The organics were dried over sodium sulfate, filtered, and concentrated to yield crude urea intermediate. The urea residue was taken into 2.2 mL of a 1.0 M malonic acid solution in acetic acid and treated with 0.66 mL of acetic anhydride. The mixture was heated to 80° C. for 5 hr with stirring. The solution was concentrated to yield the crude barbituric acid intermediate which was immediately taken into 2 mL of dimethylsulfoxide and 0.84 mL of triethylamine. To this was added 0.36 mL of carbon disulfide. The solution was stirred at 23° C. for 1 hr before 0.2 mL of 1,3-dibromopropane was added. Stirring at 23° C. was continued for 2 hr at which time ethyl acetate (5 mL) and water (3 mL) was added. Upon standing, solid dithiane intermediate 1-butyl-5-(1,3-dithian-2-ylidene)-3-{4-[(trifluoromethyl)thio]phenyl}pyrimidine-2,4,6(1H,3H,5H)-trione was isolated from the biphasic medium after filtration and drying under vacuum (320 mg, 34% yield).

Into a sealable pressure tube was prepared a suspension of 200 mg of the above dithiane intermediate in 2 mL of methanol and 4 mL of 2.0 M ammonia in methanol solution. The tube was sealed and stirred at 100° C. for 2 hr before partially cooling the resulting solution to ˜3 mL volume. Upon standing at 0° C. the partially concentrated reaction mixture did not provide any solids. The mixture was concentrated and purified by silica gel chromatography eluting with dichloromethane/ethyl acetate solvent to yield 80 mg (47% yield) of the title compound as a white solid after drying under vacuum for several hours: ¹H NMR (300 MHz, CDCl₃) δ 9.9 (br s, 2H), 7.78 (d, 2H,1J=8.3), 7.31 (d, 2H,1J=8.4), 5.4 (br s, 2H), 3.93 (t, 2H,1J=7.5), 1.7-1.6 (m, 2H), 0.96 (t, 3H, J=7.3); MS (m/z) 403.4 (MH+, 100%).

Examples 6-10 were prepared in identical fashion to that described for the preparation of Example 5.

### Example 6

### 1-Butyl-5-(diaminomethylene)-3-mesitylpyrimidine-2,4,6(1H,3H,5H)-trione

The title compound was prepared as described for example 5. Thus, 2 mmol (322 mg) of 2,4,6-trimethylphenylisocyanate gave 250 mg of intermediate 1-butyl-5-(1,3-dithian-2-ylidene)-3-mesitylpyrimidine-2,4,6(1H,3H,5H)-trione as a solid. From 200 mg of this intermediate was prepared the crude partially concentrated methanol/ammonia solution of the title compound. The solution was cooled to 0° C. and allowed to stand for several hours. The white solids were isolated by filtration washing with cold ethyl acetate and dried under vacuum for several hours to yield 120 mg of the title compound: ¹H NMR (300 MHz, CDCl₃) δ 10.2 (br s, 2H), 6.97 (s, 2H), 5.25 (br s, 2H), 3.98 (t, 2H, J=7.4), 2.32 (s, 3H), 2.09 (s, 6H), 1.71-1.58 (m, 2H), 1.43-1.35 (m, 2H), 0.96 (t, 3H, J=7.3); MS (m/z) 345.3 (MH+, 100%).

### Example 7

### 1-Butyl-5-(diaminomethylene)-3-(2,3-dihydro-1H-inden-5-yl)pyrimidine-2,4,6(1H,3H,5H)-trione

The title compound was prepared as described for example 5. Thus, 2 mmol (320 mg) of 5-isocyanatoindane gave 300 mg of intermediate 1-butyl-3-(2,3-dihydro-1H-inden-5-yl)-5-(1,3-dithian-2-ylidene)pyrimidine-2,4,6(1H,3H,5H)-trione as a solid. From 200 mg of this intermediate was prepared the crude partially concentrated methanol/ammonia solution of the title compound. The solution was cooled to 0° C. and allowed to stand for several hrs. The white solids were isolated by filtration washing with cold ethyl acetate and dried under vacuum for several hours to yield 70 mg of the title compound: ¹H NMR (300 MHz, DMSO-d6) δ 9.47 (br s, 2H), 7.34 (s, 2H), 7.24 (d, 1H, J=7.9), 7.01 (s, 1H), 6.9 (dd, 1H, J=7.9, 1.8), 3.78 (t, 2H, J=7.1), 2.81 (dd, 4H, J=12, 7.1), 2.65 (m, 2H), 1.51 (m, 2H), 1.35-1.22 (m, 2H), 0.89 (t, 3H, J=7.2); MS (m/z) 341.13 (MH+, 100%).

### Example 8

### 1-(1,3-Benzodioxol-5-yl)-3-butyl-5-(diaminomethylene)pyrimidine-2,4,6(1H,3H,5H)-trione

The title compound was prepared as described for example 5. Thus, 2 mmol (330 mg) of 5-isocyanato-1,3-benzodioxole gave 160 mg of intermediate 1-(1,3-benzodioxol-5-yl)-3-butyl-5-(1,3-dithian-2-ylidene)pyrimidine-2,4,6(1H,3H,5H)-trione as a solid. From 140 mg of this intermediate was prepared the crude partially concentrated methanol/ammonia solution of the title compound. The solution was cooled to 0° C. and allowed to stand for several hrs. The white solids were isolated by filtration washing with cold ethyl acetate and dried under vacuum for several hours to yield 75 mg of the title compound: ¹H NMR (300 MHz, CDCl₃) δ 10.1 (br d, 2H), 6.87 (dd, 1H, J=5.1, 3.1), 6.69 (d, 1H, J=2.0), 6.68 (d, 1H, 1=2.1), 5.99 (s, 2H), 5.29 (br s, 2H), 3.92 (t, 2H, J=7.6), 1.62 (m, 2H), 1.37 (m, 2H), 0.93 (t, 3H, J=7.4); MS (m/z) 347.4 (MH+, 100%).

### Example 9

### 1-Butyl-5-(diaminomethylene)-3-(2,4-difluorophenyl)pyrimidine-2,4,6(1H,3H,5H)-trione

The title compound was prepared as described for example 5. Thus, 2 mmol (310 mg) of 2,4-difluoro-1-isocyanatobenzene gave 220 mg (27% yield) of intermediate 1-butyl-3-(2,4-difluorophenyl)-5-(1,3-dithian-2-ylidene)pyrimidine-2,4,6(1H,3H,5H)-trione as a solid. From 200 mg of this intermediate was prepared the crude partially concentrated methanol/ammonia solution of the title compound. The solution was cooled to 0° C. and allowed to stand for several hrs. The white solids were isolated by filtration washing with cold ethyl acetate and dried under vacuum for several hours to yield 70 mg (43% yield) of the title compound: ¹H NMR (300 MHz, CDCl₃) δ 10.2 (br d, 2H), 7.3 (m, 1H), 7.03-6.93 (m, 2H), 5.41 (br s, 2H), 3.95 (t, 2H, J=7.5), 1.62 (m, 2H), 1.40 (m, 2H), 0.96 (t, 3H, J=7.3); MS (m/z) 339.3 (MH+, 100%).

### Example 10

### 1-Butyl-5-(diaminomethylene)-3-(2-fluorophenyl)pyrimidine-2,4,6(1H,3H,5H)-trione

The title compound was prepared as described for example 5. Thus, 2 mmol (274 mg) of 1-fluoro-2-isocyanatobenzene gave 300 mg (38% yield) of intermediate 1-butyl-5-(1,3-dithian-2-ylidene)-3-(2-fluorophenyl)pyrimidine-2,4,6(1H,3H,5H)-trione as a solid. From 200 mg of this intermediate was prepared the crude partially concentrated methanol/ammonia solution of the title compound. The solution was cooled to 0° C. and allowed to stand for several hrs. The white solids were isolated by filtration washing with cold ethyl acetate and dried under vacuum for several hours to yield 140 mg (87% yield) of the title compound: MS (m/z) 321.3 (MH+, 100%).

### Example 11

### 1-Butyl-5-(diaminomethylene)-3-(4-phenylbutyl)pyrimidine-2,4,6(1H,3H,5H)-trione

### 11(a) Preparation of intermediate 1-butyl-5-(1,3-dithian-2-ylidene)-3-(4-phenylbutyl)pyrimidine-2,4,6(1H,3H,5H)-trione

To an ice-water cooled solution of butylisocyanate (0.32 g, 3.2 mmol) in dichloromethane (10 mL) was added dropwise a solution of 4-phenylbutylamine (0.48 g, 3.2 mmol, 1.0 eq) in dichloromethane (10 mL) with stirring under nitrogen. The ice-water bath was removed and the reaction mixture was stirred at room temperature under nitrogen overnight. The reaction mixture was concentrated in vacuo to give 0.77 g of the crude urea as a pale yellow oil which solidified upon standing to give a white solid. To the crude urea (0.77 g) were added malonic acid (0.34 g, 3.27 mmoL), acetic anhydride (2 mL, 2.16 g, 21.2 mmoL), and acetic acid (3 mL). The reaction mixture was heated at 80° C. with stirring under nitrogen for 6.5 h. The reaction mixture was concentrated in vacuo to give 1.10 g of the crude barbituric acid as an orange oil. To a solution of the crude barbituric acid (0.345 g) in dimethylsulfoxide (2 mL) was added triethylamine (0.46 mL, 0.334 g, 3.30 mmoL). The solution was cooled in an ice-water bath and carbon disulfide (0.20 mL, 0.252, g, 3.31 mmoL) was slowly added to the reaction mixture with stirring under nitrogen. The ice-water bath was removed and the reaction mixture was allowed to stir at room temperature for 2 h. 1,3-Dibromopropane (0.11 mL, 0.219 g, 1.08 mmoL) was added to the reaction mixture and the reaction mixture was stirred under nitrogen for 1 h. The reaction mixture was partitioned between water and EtOAc and the organic phase was washed with water and brine. The organic phase was dried over MgSO₄, filtered, and the filtrate was concentrated under reduced pressure to give 0.57 g of the crude product. The crude product was purified by flash chromatography (SiO₂) with CH₂Cl₂ as eluant to give 0.177 g (41% yield based on 1.0 mmoL of butylisocyanate) of intermediate 1-butyl-5-(1,3-dithian-2-ylidene)-3-(4-phenylbutyl)pyrimidine-2,4,6(1H,3H,5H)-trione as a yellow oil. ¹H NMR (400 MHz, CDCl₃): δ 0.93 (t, 3H, J=7.3 Hz), 1.36 (m, 2H), 1.58-1.64 (m, 2H), 1.67 (m, 4H), 2.35 (m, 2H), 2.64 (m, 2H), 2.96 (t, 4H, J=7.1 Hz), 3.94 (m, 4H), 7.16 (m, 3H), 7.26 (m, 2H).

### 11(b) Preparation of 1-butyl-5-(diaminomethylene)-3-(4-phenylbutyl)pyrimidine-2,4,6(1H,3H,5H)-trione

To a glass, sealable pressure tube were added 1-butyl-5-(1,3-dithian-2-ylidene)-3-(4-phenylbutyl)pyrimidine-2,4,6(1H,3H,5H)-trione (0.039 g, 0.09 mmoL) and 2 M ethanolic ammonia (3 mL). The bottle was capped and the reaction mixture was heated at 83° C. for 15 h. The reaction mixture was concentrated in vacuo to give 0.034 of the crude product. The crude product was purified by flash chromatography over SiO₂ with CH₂Cl₂:MeOH (98:2) as eluant to give 19 mg (59% yield) of the title compound as a white solid. ¹H NMR (400 MHz, CDCl₃): δ 0.93 (t, 3H, J=7.3 Hz), 1.36 (m, 2H), 1.57-1.62 (m, 2H), 1.66 (m, 4H), 2.64 (m, 2H), 3.91 (m, 4H), 5.10 (br s, 2H), 7.16 (m, 3H), 7.26 (m, 2H), 10.38 (br s, 2H). Electrospray MS (m/z) 359.2 (MH+, 100%). HRMS (M+H)⁺ Calc: 359.2083; Found: 359.2072.

### Example 12

### 1-Butyl-3-cyclohexyl-5-(diaminomethylene)pyrimidine-2,4,6(1H,3H,5H)-trione

### 12(a) Preparation of Intermediate 1-butyl-3-cyclohexyl-5-(1,3-dithian-2-ylidene)pyrimidine-2,4,6(1H,3H,5H)-trione

To a solution of butylisocyanate (0.074 g, 0.75 mmoL) in 1,2-dichloroethane (2 mL) was added a solution of cyclohexylamine (0.08 g, 0.81 mmoL, 1.08 eq) in dichloroethane (2 mL) at room temperature with stirring. The reaction mixture was stirred at room temperature overnight. The reaction mixture was concentrated in vacuo to give the crude urea. To the crude urea were added malonic acid (0.078 g, 0.75 mmoL), acetic anhydride (0.5 mL, 0.54 g, 5.3 mmoL), and acetic acid (0.75 mL). The reaction mixture was heated at 80° C. for 6 h. The reaction mixture was concentrated in vacuo and 1 mL of toluene was added. The solvent was removed in vacuo to give the crude barbituric acid. To the crude barbituric acid was added DMSO (1.4 mL) and triethylamine (0.42 mL, 0.30 g, 3 mmoL) followed by carbon disulfide (0.14 mL, 0.18 g, 2.3 mmoL). The reaction mixture was stirred at room temperature for 2 h. To the reaction mixture was added 1,3-dibromopropane (0.075 mL, 0.15 g, 0.74 mmoL). The reaction was stirred at room temperature for 1 h. The reaction mixture was partitioned between water and EtOAc. The organic phase was separated and concentrated in vacuo to give the crude product. The crude product was purified by flash chromatography over SiO₂ with CH₂Cl₂:Hexanes (2:1) as eluant to give 0.222 g (77%) of intermediate 1-butyl-3-cyclohexyl-5-(1,3-dithian-2-ylidene)pyrimidine-2,4,6(1H,3H,5H)-trione as a yellow solid. ¹H NMR (300 MHz, CDCl₃): δ 0.96 (t, 3H, J=7.3 Hz), 1.23-1.47 (m, 5H), 1.60-1.72 (m, 5H), 1.85 (m, 2H), 2.32-2.44 (m, 4H), 2.97 (t, 4H, J=7.0 Hz), 3.92 (m, 2H), 4.77 (tm, 1H, J=12.3 Hz)

### 12(b) Preparation of 1-butyl-3-cyclohexyl-5-(diaminomethylene)pyrimidine-2,4,6(1H,3H,5H)-trione

1-butyl-3-cyclohexyl-5-(1,3-dithian-2-ylidene)pyrimidine-2,4,6(1H,3H,5H)-trione (0.070 g, 0.18 mmoL) and 2 M ethanolic ammonia (4 mL) were combined in a pressure tube. The bottle was sealed with a screw-cap and the reaction mixture was heated at 80° C. overnight. The reaction mixture was concentrated in vacuo to give the crude product. The crude product was purified by flash chromatography over SiO₂ with CH₂Cl₂:MeOH (95:5) as eluant to give 0.048 g (86%) of the title compound as a white solid. ¹H NMR (400 MHz, CDCl₃): δ 93 (t, 3H, J=7.4 Hz), 1.23 (tm, 1H, J=12.8 Hz), 1.30-1.41 (m, 4H), 1.55-1.68 (m, 5H), 1.82 (m, 2H), 2.38 (qd, 2H, J=12.5, 3.4 Hz), 3.87 (m, 2H), 4.75 (tt, 1H, J=12.2, 3.7 Hz), 5.06 (br s, 2H), 10.42 (br s, 2H). HRMS (M+H)⁺Calc: 309.1927; Found: 309.1920.

### Example 13

### 1-Butyl-3-(cyclohexylmethyl)-5-(diaminomethylene)pyrimidine-2,4,6(1H,3H,5H)-trione

### 13(a) Preparation of Intermediate 1-butyl-3-(cyclohexylmethyl)-5-(1,3-dithian-2-ylidene)pyrimidine-2,4,6(1H,3H,5B)-trione

To a solution of butylisocyanate (0.074 g, 0.75 mmoL) in 1,2-dichloroethane (2 mL) was added a solution of cyclohexanemethylamine (0.095 g, 0.84 mmoL, 1.12 eq) in dichloroethane (2 mL) at room temperature. The reaction mixture was stirred at room temperature overnight. The reaction mixture was concentrated in vacuo to give the crude urea. To the crude urea were added malonic acid (0.078 g, 0.75 mmoL), acetic anhydride (0.5 mL, 0.54 g, 5.3 mmoL), and acetic acid (0.75 mL). The reaction mixture was heated at 80° C. for 6 h. The reaction mixture was concentrated in vacuo and 1 mL of toluene was added. The solvent was removed in vacuo to give the crude barbituric acid. To the crude barbituric acid were added DMSO (1.4 mL) and triethylamine (0.42 mL, 0.30 g, 3 mmoL) followed by carbon disulfide (0.14 mL, 0.18 g, 2.3 mmoL). The reaction mixture was stirred at room temperature for 2 h. To the reaction mixture was added 1,3-dibromopropane (0.075 mL, 0.15 g, 0.74 mmoL). The reaction was stirred at room temperature for 1 h. The reaction mixture was partitioned between water and EtOAc. The organic phase was separated and concentrated in vacuo to give the crude product. The crude product was purified by flash chromatography over SiO₂ with CH₂Cl₂:Hexanes (2:1) as eluant to give 0.164 g (55%) of intermediate 1-butyl-3-(cyclohexylmethyl)-5-(1,3-dithian-2-ylidene)pyrimidine-2,4,6(1H,3H,5H)-trione as a yellow solid: ¹H NMR (300 MHz, CDCl₃): δ 0.96 (t, 3H, J=7.3 Hz), 1.06 (m, 2H), 1.20 (m, 3H), 1.39 (m, 2H), 1.58-1.88 (m, 8H), 2.38 (m, 2H), 2.98 (t, 4H, J=7.1 Hz), 3.82 (d, 2H, J=7.2 Hz), 3.95 (m, 2H).

### 13(b) Preparation of 1-butyl-3-(cyclohexylmethyl)-5-(diaminomethylene)pyrimidine-2,4,6(1H,3H,5H)-trione

1-Butyl-3-(cyclohexylmethyl)-5-(1,3-dithian-2-ylidene)pyrimidine-2,4,6(1H,3H,5H)-trione (0.117 g, 0.30 mmoL) and 2 M ethanolic ammonia (4 mL) were combined in a pressure tube. The bottle was sealed with a screw-cap and the reaction mixture was heated at 80° C. overnight. The reaction mixture was concentrated in vacuo to give the crude product. The crude product was purified by flash chromatography over SiO₂ with CH₂Cl₂:CH₃OH (95:5) as eluant to give 0.020 g (21% yield) of the title compound as a white solid. ¹H NMR (300 MHz, d₆-DMSO): δ 0.88 (t, 3H, J=7.3 Hz), 0.94 (m, 2H), 1.06-1.19 (m, 3H), 1.25 (m, 2H), 1.40-1.73 (m, 8H), 3.63 (d, 2H, J=7.0 Hz), 3.76 (m, 2H), 7.31 (br s, 2H), 9.54 (br s, 2H). Electrospray MS (m/e) 323.5 (MH+, 72%). HRMS (M+H)⁺ calc: 323.2083; Found: 323.2066.

### Example 14

### 1-Butyl-3-cyclopentyl-5-(diaminomethylene)pyrimidine-2,4,6(1H,3H,5H)-trione

### 14(a) Preparation of Intermediate 1-butyl-3-cyclopentyl-5-(1,3-dithian-2-ylidene)pyrimidine-2,4,6(1H,3H,5H)-trione

To a solution of butylisocyanate (0.074 g, 0.75 mmoL) in 1,2-dichloroethane (2 mL) was added a solution of cyclopentylamine (0.070 g, 0.82 mmoL, 1.09 eq) in dichloroethane (2 mL) at room temperature. The reaction mixture was stirred at room temperature overnight. The reaction mixture was concentrated in vacuo to give the crude urea. To the crude urea were added malonic acid (0.078 g, 0.75 mmoL), acetic anhydride (0.5 mL, 0.54 g, 5.3 mmoL), and acetic acid (0.75 mL). The reaction mixture was heated at 80° C. for 6 h. The reaction mixture was concentrated in vacuo and 1 mL of toluene was added. The solvent was removed in vacuo to give the crude barbituric acid. To the crude barbituric acid was added DMSO (1.4 mL) and triethylamine (0.42 mL, 0.30 g, 3 mmoL) followed by carbon disulfide (0.14 mL, 0.18 g, 2.3 mmoL). The reaction mixture was stirred at room temperature for 2 h. To the reaction mixture was added 1,3-dibromopropane (0.075 mL, 0.15 g, 0.74 mmoL). The reaction mixture was stirred at room temperature for 1 h. The reaction mixture was partitioned between water and EtOAc. The organic phase was separated and concentrated in vacuo to give the crude product. The crude product was purified by flash chromatography over SiO₂ with CH₂Cl₂:Hexanes (2:1) as eluant to give 0.116 g (42%) of intermediate 1-butyl-3-cyclopentyl-5-(1,3-dithian-2-ylidene)pyrimidine-2,4,6(1H,3H,5H)-trione as a yellow solid. ¹H NMR (300 MHz, CDCl₃): δ 0.96 (t, 3H, J=7.3 Hz), 1.39 (m, 2H), 1.62 (m, 4H), 1.80-2.18 (m, 6H), 2.37 (m, 2H), 2.98 (t, 4H, J=7.1 Hz), 3.93 (mH, 2), 5.31 (quin, 1H, J=8.5 Hz).

### 14(b) 1-Butyl-3-cyclopentyl-5-(diaminomethylene)pyrimidine-2,4,6(1H,3H,5H)-trione

1-Butyl-3-cyclopentyl-5-(1,3-dithian-2-ylidene)pyrimidine-2,4,6(1H,3H,5H)-trione (0.079 g, 0.21 mmoL) and 2 M ethanolic ammonia (4 mL) were combined in a pressure tube. The bottle was sealed with a screw-cap and the reaction mixture was heated at 80° C. overnight. The reaction mixture was concentrated in vacuo to give the crude product. The crude product was purified by flash chromatography over SiO₂ with CH₂Cl₂:CH₃OH (95:5) as eluant to give 0.068 g of the title compound as a pale yellow solid. ¹H NMR indicates that the title compound contains an impurity. ¹H NMR (300 MHz, d₆-DMSO): δ 0.88 (t, 3H, J=7.2 Hz), 1.26 (m, 2H), 1.40-1.58 (m, 4H), 1.62-1.76 (m, 2H), 1.78-2.08 (m, 4H), 3.75 (m, 2H), 5.23 (quin, 1H, J=8.6 Hz), 7.31 (br s, 2H), 9.55 (br s, 2H); HRMS (M+H)⁺ Calc: 295.1770; Found: 295.1767.

### Example 15

### 1-Butyl-3-cycloheptyl-5-(diaminomethylene)pyrimidine-2,4,6(1H,3H,5H)-trione

### 15(a) Preparation of intermediate 1-butyl-3-cycloheptyl-5-(1,3-dithian-2-ylidene)pyrimidine-2,4,6(1H,3H,5H)-trione

To a solution of butylisocyanate (0.074 g, 0.75 mmoL) in 1,2-dichloroethane (2 mL) was added a solution of cycloheptylamine (0.091 g, 0.80 mmoL, 1.07 eq) in dichloroethane (2 mL) at room temperature. The reaction mixture was stirred at room temperature overnight. The reaction mixture was concentrated in vacuo to give the crude urea. To the crude urea were added malonic acid (0.078 g, 0.75 mmoL), acetic anhydride (0.5 mL, 0.54 g, 5.3 mmoL), and acetic acid (0.75 mL). The reaction mixture was heated at 80° C. for 6 h. The reaction mixture was concentrated in vacuo and 1 mL of toluene was added. The solvent was removed in vacuo to give the crude barbituric acid. To the crude barbituric acid were added DMSO (1.4 mL) and triethylamine (0.42 mL, 0.30 g, 3 mmoL) followed by carbon disulfide (0.14 mL, 0.18 g, 2.3 mmoL). The reaction mixture was stirred at room temperature for 2 h. To the reaction mixture was added 1,3-dibromopropane (0.075 mL, 0.15 g, 0.74 mmoL). The reaction mixture was stirred at room temperature for 1 h. The reaction mixture was partitioned between water and EtOAc. The organic phase was separated and concentrated in vacuo to give the crude product. The crude product was purified by flash chromatography over SiO₂ with CH₂Cl₂:Hexanes (2:1) as eluant to give 0.181 g (61% yield) of intermediate 1-butyl-3-cycloheptyl-5-(1,3-dithian-2-ylidene)pyrimidine-2,4,6(1H,3H,5H)-trione as a yellow solid. ¹H NMR (300 MHz, CDCl₃): δ 0.95 (t, 3H, J=7.3 Hz), 1.38 (m, 2H), 1.47-1.70 (m, 8H), 1.70-1.87 (m, 4H), 2.28-2.44 (m, 4H), 2.97 (t, 4H, J=7.1 Hz), 3.92 (m, 2H), 4.91 (m, 1H).

### 15(b) Preparation of 1-butyl-3-cycloheptyl-5-(diaminomethylene)pyrimidine-2,4,6(1H,3H,5H)-trione

1-Butyl-3-cycloheptyl-5-(1,3-dithian-2-ylidene)pyrimidine-2,4,6(1H,3H,5H)-trione, (0.153 g, 0.39 mmoL) and 2 M ethanolic ammonia (4 mL) were combined in a pressure tube. The bottle was sealed with a screw-cap and the reaction mixture was heated at 80° C. overnight. The reaction mixture was concentrated in vacuo to give the crude product. The crude product was purified by flash chromatography over SiO₂ with CH₂Cl₂:CH₃OH (97:3) as eluant to give 0.058 g (46% yield) of the title compound as a pale yellow solid. ¹H NMR (300 MHz, d₆-DMSO): δ 0.87 (t, 3H, J=7.1 Hz), 1.18-1.78 (m, 14H), 2.22 (m, 2H), 3.74 (m, 2H), 4.82 (m, 1H), 7.28 (br s, 2H), 9.55 (br s, 2H); HRMS (M+H)⁺ Calc: 323.2083, Found: 323.2064.

### Example 16

### 1-Butyl-3-cyclooctyl-5-(diaminomethylene)pyrimidine-2,4,6(1H,3H,5H)-trione

### 16(a) Preparation of Intermediate 1-butyl-3-cyclooctyl-5-(1,3-dithian-2-ylidene)pyrimidine-2,4,6(1H,3H,5H)-trione

To a solution of butylisocyanate (0.074 g, 0.75 mmoL) in 1,2-dichloroethane (2 mL) was added a solution of cyclooctylamine (0.098 g, 0.77 mmoL, 1.03 eq) in dichloroethane (2 mL) at room temperature. The reaction mixture was stirred at room temperature overnight. The reaction mixture was concentrated in vacuo to give the crude urea. To the crude urea were added malonic acid (0.078 g, 0.75 mmoL), acetic anhydride (0.5 mL, 0.54 g, 5.3 mmoL), and acetic acid (0.75 mL). The reaction mixture was heated at 80° C. for 6 h. The reaction mixture was concentrated in vacuo and 1 mL of toluene was added. The solvent was removed in vacuo to give the crude barbituric acid. To the crude barbituric acid was added DMSO (1.4 mL) and triethylamine (0.42 mL, 0.30 g, 3 mmoL) followed by carbon disulfide (0.14 mL, 0.18 g, 2.3 mmoL). The reaction mixture was stirred at room temperature for 2 h. To the reaction mixture was added 1,3-dibromopropane (0.075 mL, 0.15 g, 0.74 mmoL). The reaction was stirred at room temperature for 1 h. The reaction mixture was partitioned between water and EtOAc. The organic phase was separated and concentrated in vacuo to give the crude product. The crude product was purified by flash chromatography over SiO₂ with CH₂Cl₂:Hexanes (2:1) as eluant to give 0.192 g (62% yield) of intermediate 1-butyl-3-cyclooctyl-5-(1,3-dithian-2-ylidene)pyrimidine-2,4,6(1H,3H,5H)-trione as a yellow oil. ¹H NMR (300 MHz, CDCl₃): δ 0.95 (t, 3H, J=7.3 Hz), 1.38 (m, 2H), 1.47-1.88 (m, 14H), 2.28-2.43 (m, 4H), 2.97 (t, 4H, J=7.1 Hz), 3.92 (m, 2H), 5.05 (m, 1H).

### 16(b) Preparation of 1-butyl-3-cyclooctyl-5-(diaminomethylene)pyrimidine-2,4,6(1H,3H,5H)-trione

1-Butyl-3-cyclooctyl-5-(1,3-dithian-2-ylidene)pyrimidine-2,4,6(1H,3H,5H)-trione (0.160 g, 0.39 mmoL) and 2 M ethanolic ammonia (5 mL) were combined in a pressure tube. The bottle was sealed with a screw-cap and the reaction mixture was heated at 80° C. overnight. The reaction mixture was concentrated in vacuo to give the crude product. The crude product was purified by flash chromatography over SiO₂ with CH₂Cl₂:CH₃OH (99:1) as eluant to give 0.112 g (85% yield) of the title compound as an off-white solid. ¹H NMR (400 MHz, d₆-DMSO): δ 0.85 (t, 3H, J=7.4 Hz), 1.22 (m, 2H), 1.34-1.61 (m, 11H), 1.62-1.73 (m, 3H), 2.20 (m, 2H), 3.71 (m, 2H), 4.73-5.10 (m, 1H), 7.25 (br s, 2H), 9.53 (br s, 2H); HRMS (M+H)+Calc: 337.2240, Found: 337.2226.

### Example 17

### 1-Butyl-5-(diaminomethylene)-3-(3-phenylcyclopentyl)pyrimidine-2,4,6(1H,3H,5H)-trione

17(a) Preparation of intermediate 1-butyl-5-(1,3-dithian-2-ylidene)-3-(3-phenylcyclopentyl)pyrimidine-2,4,6(1H,3H,5H)-trione: To a solution of 3-phenylcyclopentylamine maleate (0.249 g, 0.90 mmoL), triethylamine (0.376 mL, 0.273 g, 2.70 mmoL, 3 eq) in dichloromethane (4 mL) was slowly added a solution of butylisocyanate (0.101 mL, 0.089 g, 0.90 mmoL, 1.0 eq) in dichloromethane (1 mL). The reaction mixture was shaken at room temperature overnight. The reaction mixture was partitioned between 1 N aqueous HCl and dichloromethane. The layers were separated and the organic phase was dried over MgSO₄, filtered, and the filtrate was concentrated to give 0.27 g of the crude urea. The crude urea was combined with malonic acid (0.105 g, 1.0 mmoL), acetic anhydride (0.283 mL, 0.30 g, 3.0 mmoL), and acetic acid (2 mL). The reaction mixture was heated at 80° C. with stirring for 6 h. The reaction mixture was concentrated in vacuo to give the crude barbituric acid. To the crude barbituric acid were added DMSO (2.4 mL) and triethylamine (0.42 mL, 0.30 g, 3.0 mmoL). To the solution was added carbon disulfide (0.183 mL, 0.23 g, 3.0 mmoL) at room temperature. The reaction mixture was agitated for 2 h. 1,3-Dibromopropane (0.10 mL, 0.2 g, 0.99 mmoL) was added and the reaction mixture was agitated for 1 h. The reaction mixture was partitioned between water and EtOAc. The layers were separated and the organic phase was dried over MgSO₄, filtered, and the filtrate was concentrated to give the crude product. The crude product was purified by flash chromatography over SiO₂ with hexanes:ethyl acetate (2:1) to give 0.06 g (15% yield) of intermediate 1-butyl-5-(1,3-dithian-2-ylidene)-3-(3-phenylcyclopentyl)pyrimidine-2,4,6(1H,3H,5H)-trione as a yellow oil. ¹H NMR (400 MHz, CDCl₃): δ 0.94 (t, 3H, J=7.4 Hz), 1.38 (m, 2H), 1.59-1.74 and 1.97-2.49 (m, 10H) (diastereomers), 2.96 (t, 4H, J=7.2 Hz), 3.05 and 3.71 (m, 1H) (diastereomers), 3.93 (m, 2H), 5.44-5.62 (m, 1H), 7.15-7.21, (m, 1H), 7.27-7.35 (m, 4H).

### 17(b) Preparation of 1-butyl-5-(diaminomethylene)-3-(3-phenylcyclopentyl)pyrimidine-2,4,6(1H,3H,5H)-trione

1-Butyl-5-(1,3-dithian-2-ylidene)-3-(3-phenylcyclopentyl)pyrimidine-2,4,6(1H,3H,5H)-trione (0.06 g, 0.13 mmoL) and 2 M ethanolic ammonia (5 mL) were combined in a pressure tube. The glass tube was sealed with a screw cap and the reaction mixture was heated at 80° C. for 6 h. The reaction mixture was concentrated in vacuo to give the crude product. The crude product was purified by flash chromatography over SiO₂ with CH₂Cl₂:CH₃OH (99:1 to 98:2) as eluant to give 0.026 g (54% yield) of the title compound as a pale yellow solid. ¹H NMR (400 MHz, CDCl₃): δ 0.94 (t, 3H, J=7.3 Hz), 1.38 (m, 2H), 1.57-1.76 and 1.94-2.53 (m, 811 (diastereomers), 3.04 and 3.70 (m, 1H) (diastereomers), 3.90 (m, 2H), 5.07 (br s, 2H), 5.46-5.62 (m, 1H), 7.16-7.21 (m, 1H), 7.26-7.36 (m, 4H), 10.43 (br s, 2H); HRMS (M+Na)⁺ Calc: 393.1903; Found 393.1931.

### Example 18

### 1-Butyl-5-(diaminomethylene)-3-(5-phenylpentyl)pyrimidine-2,4,6(1H,3H,5H)-trione

Preparation of 1-butyl-5-(diaminomethylene)-3-(5-phenylpentyl)pyrimidine-2,4,6(1H,3H,5H)-trione: To an agitated solution of 5-phenylpentylamine hydrochloride (0.080 g, 0.40 mmoL) and triethylamine (0.084 mL, 0.0.061 g, 0.60 mmoL, 1.5 eq) in dichloromethane (4 mL) was slowly added a solution of butylisocyanate (0.045 mL, 0.040 g, 0.40 mmoL, 1.0 eq) in dichloromethane (1 mL). The reaction mixture was agitated on a shaker table at room temperature overnight. The reaction mixture was partitioned between 1 N aqueous HCl and dichloromethane. The layers were separated and the organic phase was dried over MgSO₄, filtered, and the filtrate was concentrated to give 0.076 g of the urea. The urea (0.070 g, 0.26 mmoL) was combined with malonic acid (0.025 g, 0.24 mmoL), acetic anhydride (0.065 mL, 0.070 g, 0.69 mmoL), and acetic acid (2 mL). The reaction mixture was heated at 80° C. for 6 h. The reaction mixture was concentrated in vacuo to give the crude barbituric acid intermediate. To the crude barbituric acid intermediate were added DMSO (2 mL) and triethylamine (0.96 mL, 0.70 g, 6.9 mmoL). To the solution was added carbon disulfide (0.042 mL, 0.053 g, 0.70 mmoL). The reaction mixture was shaken on a shaker table at room temperature for 2 h. 1,3-Dibromopropane (0.024 mL, 0.048 g, 0.24 mmoL) was added and the reaction mixture was agitated on a shaker table for 1 h. The reaction mixture was partitioned between water and EtOAc. The layers were separated and the organic phase was dried over MgSO₄, filtered, and the filtrate was concentrated in vacuo to give the crude product. The crude product was purified by flash chromatography over SiO₂ with hexanes:ethyl acetate (3:1) to give 0.011 g of intermediate 1-butyl-5-(1,3-dithian-2-ylidene)-3-(5-phenylpentyl)pyrimidine-2,4,6(1H,3H,5H)-trione. To this intermediate was added 2 M ethanolic ammonia (5 mL) and the reaction mixture was heated in a pressure tube at 80° C. for 6 h. The crude product was purified by flash chromatography over SiO₂ with CH₂Cl₂:CH₃OH (99:1 to 98:2) as eluant to give 0.002 g (2% yield based on 0.24 mmoL of malonic acid) of the title compound as a white solid. ¹H NMR (300 MHz, CDCl₃): δ 0.96 (t, 3H, J=7.3 Hz), 1.35-1.48 (m, 4H), 1.62-1.75 (m, 6H), 2.63 (m, 2H), 3.92 (m, 4H), 5.11 (br s, 2H), 7.15-7.24 (m, 3H), 7.25-7.32 (m, 2H), 10.41 (br s, 2H); HRMS (M+Na)⁺ calcd: 395.2059, Found: 395.2068.

### Example 19

### 1-[3-(Benzyloxy)phenyl]-3-butyl-5-(diaminomethylene)pyrimidine-2,4,6(1H,3H,5H)-trione

The title compound was prepared identical to that described for the preparation of example 3. Thus, 0.50 mmol of 3-benzyloxyaniline gave 56 mg (23% yield) of intermediate 1-[3-(benzyloxy)phenyl)-3-butyl-5-(1,3-dithian-2-ylidene)pyrimidine-2,4,6(1H,3H,5H)-trione after chromatography as described. From 50 mg of this dithiane intermediate was obtained 12 mg (28% yield) of the title compound as a white solid: ¹H NMR (400 MHz, CDCl₃) δ 10.08 (bs, 1H), 9.82 (bs, 1H), 7.45-7.30 (m, 6H), 7.02 (d, 1H, J=8.4), 6.88-6.79 (m, 2H), 5.42 (bs, 2H), 5.02 (s, 2H), 3.91 (t, 2H, J=6.8), 1.68-1.57 (m, 2H), 1.44-1.35 (m, 2H), 0.92 (t, 3H, J=7.2); MS (m/z) 377.4 (MH+ 24%), 399.4 (M+Na 100%).

### Example 20

### Benzyl 3-[3-butyl-5-(diaminomethylene)-2,4,6-trioxotetrahydropyrimidin-1(2H)-yl]propylcarbamate

The title compound was prepared identical to that described for the preparation of example 3. Thus, 25.1 mmol of benzyl 3-aminopropylcarbamate gave 2.30 g (19% yield) of intermediate benzyl 3-[3-butyl-5-(1,3-dithian-2-ylidene)-2,4,6-trioxotetrahydropyrimidin-1(2H)-yl]propylcarbamate after chromatography as described. From 2.29 g of this dithiane intermediate was obtained 1.45 g (75% yield) of the title compound as a white solid: ¹H NMR (400 MHz, CDCl₃) δ 10.05 (bs, 2H), 7.41-7.26 (m, 5H), 6.10-5.80 (m, 2H), 5.12 (s, 2H), 4.02-3.92 (m, 2H), 3.92-3.85 (m, 2H), 3.20-3.08 (m, 2H), 1.88-1.80 (m, 2H), 1.62-1.55 (m, 2H), 1.41-1.32 (m, 2H), 0.92 (t, 3H, J=7.2); MS (m/z) 418.4 (MH+ 9%), 440.3 (M+Na 100%).

### Example 21

### 4-Nitrobenzyl 3-[3-butyl-5-(diaminomethylene)-2,4,6-trioxotetrahydropyrimidin-1(2H)-yl]propylcarbamate

### Preparation of intermediate 1-(3-aminopropyl)-3-butyl-5-(diaminomethylene)pyrimidine-2,4,6(1H, 3H, 5H)-trione

To a solution of 1.43 g (3.42 mmol) of benzyl 3-[3-butyl-5-(diaminomethylene)-2,4,6-trioxotetrahydropyrimidin-1(2H)-yl]propylcarbamate (example 20) in 25 mL of chloroform and 10 mL of methanol was added 200 mg of 10% Pd/C and the mixture was shaken under 10 psi 11, for 16 hr. The solution was filtered through Celite and evaporated to give 0.95 g (97% yield) of intermediate 1-(3-aminopropyl)-3-butyl-5-(diaminomethylene)pyrimidine-2,4,6(1H, 3H, 5H)-trione as a white solid: ¹H NMR (400 MHz, DMSO-d₆) δ 9.44 (s, 2H), 7.97 (bs, 2H), 7.65 (s, 2H), 3.80 (t, 2H, J=6.4), 3.73 (t, 2H, J=7.2), 2.78-2.66 (m, 2H), 1.82-1.78 (m, 2H), 1.48-1.39 (m, 2H), 1.30-1.18 (m, 2H), 0.85 (t, 3H, J=7.2); MS (17z/z) 284.5 (MH+ 100%).

To a solution of 40 mg (0.14 mmol) of the above intermediate 1-(3-aminopropyl)-3-butyl-5-(diaminomethylene)pyrimidine-2,4,6(1H, 3H, 5H)-trione and 41 μL of TEA in 1 mL DMF at 0° C. was added 32 mg (0.15 mmol) of 4-nitrobenzyl chloroformate. After stirring at 23° C. for 1 hr, the mixture was diluted with ethyl acetate (20 mL) then washed with three portions of water (10 mL) and brine (10 mL), dried over Na₂SO₄ and evaporated to give 43 mg (66% yield) of the title compound as a white solid: ¹H NMR (400 MHz, CDCl₃) δ 10.10 (bs, 2H), 8.19 (d, 2H, J=8.4),7.49 (d, 2H, J=8.4), 5.86 (t, 2H, J=6.0), 5.80 (bs, 2H), 5.19 (s, 2H), 3.98 (t, 2H, J=6.4), 3.87 (t, 2H, J=7.6), 3.21-3.15 (m, 2H), 1.86-1.79 (m, 2H), 1.62-1.55 (m, 2H), 1.41-1.29 (m, 2H), 0.92 (t, 3H, J=7.2); MS (m/z) 463.1 (MH+ 100%).

### Example 22

### 4-Fluorobenzyl 3-[3-butyl-5-(diaminomethylene)-2,4,6-trioxotetrahydropyrimidin-1(2H)-yl]propylcarbamate

The title compound was prepared in the same way described for the preparation of example 21. Thus, 20 mg (0.11 mmol) of 4-fluorobenzyl chloroformate (Chem. Pharm. Bull. 1988, 36 (11), 4426-4434) gave 31 mg (67%) of the title compound as a white solid: ¹H NMR (400 MHz, CDCl₃) δ 10.15 (bs, 2H), 7.39-7.30 (m, 2H), 7.08-7.00 (m, 2H), 5.82-5.60 (m, 2H), 5.40 (s, 2H), 3.97 (t, 2H, J=6-4), 3.87 (t, 2H, J=7.6), 3.19-3.08 (m, 2H), 1.86-1.78 (m, 2H), 1.60-1.52 (m, 2H), 1.39-1.29 (m, 2H), 0.93 (t, 3H, J=7.2).

### Example 23

### 4-(2λ⁵-Triaza-1,2-dienyl)benzyl 3-[3-butyl-5-(diaminomethylene)-2,4,6-trioxotetrahydropyrimidin-1(2B)-yl]propylcarbamate

The title compound was prepared in the same way described for the preparation of example 21. Thus, 48 mg (0.15 mmol) of 4-nitrophenyl 4-(2λ⁵-triaza-1,2-dienyl)benzyl carbonate (J. Chem. Soc. Perkin Trans. I 1996, 11, 1205-1212) gave 10 mg (12% yield) of the title compound as a white solid: ¹H NMR (400 MHz, DMSO-d₆) δ 9.45 (bs, 2H), 7.39-7.25 (m, 4H), 7.21-7.16 (m, 1H), 7.08 (d, 2H, J=8.0), 4.97 (s, 2H), 3.78-3.65 (m, 4H), 2.99-2.92 (m, 2H), 1.65-1.56 (m, 2H), 1.51-1.40 (m, 2H), 1.30-1.20 (m, 2H), 0.85 (t, 3H, J=7.2); MS (m/z) 459.2 (MH+ 11%), 481.2 (M+Na 100%).

### Example 24

### 1-But-3-enyl-3-cyclopentyl-5-(diaminomethylene)pyrimidine-2,4,6(1H,3H,5H)-trione

### Preparation of Intermediate 1-but-3-enyl-3-cyclopentyl-5-(1,3-dithian-2-ylidene)pyrimidine-2,4,6-(1H, 3H, 5H)-trione

To 340 mg (3.35 mmol) of malonic acid in acetic anhydride (2 mL) and acetic acid (1 mL) was added 610 mg (3.35 mmol) of N-but-3-enyl-N′-cyclopentylurea and the mixture heated to 80° C. for 1.5 hr. Upon cooling the solution was concentrated and dried in vacu to give the crude barbituric acid which was then taken into DMSO (7 mL) before adding TEA (1.85 mL, 13.3 mmol) and CS₂ (600 μL, 9.95 mmol). The mixture was stirred for 1 hr then 1,3-dibromopropane (370 μL, 3.65 mmol) was added. After 16 hr at 23° C., ethyl acetate (50 mL) was added and the solution was washed three times with water (35 mL), then brine (35 mL) and dried over Na₂SO₄. The solution was concentrated and purified by silica gel chromatography eluting with hexanes/ethyl acetate solvent to give 550 mg (45% yield) of intermediate 1-but-3-enyl-3-cyclopentyl-5-(1,3-dithian-2-ylidene)pyrimidine-2,4,6-(1H, 3H, 5H)-trione as an orange solid: ¹H NMR (400 MHz, CDCl₃) δ 5.79-5.72 (m, 1H), 5.35-5.20 (m, 1H), 5.11-4.98 (m, 2H), 3.99 (t, 2H, J=7.6), 2.95 (t, 4H, J=7.2), 2.44-2.29 (m, 4H), 2.12-2.02 (m, 2H), 2.02-1.88 (m, 2H), 1.88-1.77 (m, 2H), 1.65-1.51 (m, 2H); MS (m/z) 367.1 (MH+ 100%).

Into a sealable pressure tube was prepared a suspension of 550 mg of the above intermediate in 2.0M N in methanol solution (10 mL.) The tube was sealed and the contents stirred at 110° C. for 2.5 hr. The solution was concentrated and the residue purified by silica gel chromatography eluting with hexanes/ethyl acetate solvent to give 405 mg (92% yield) of the title compound as a white solid: ¹H NMR (400 MHz, CDCl₃) δ 10.21 (bs, 2H), 5.87-5.45 (m, 3H), 5.38-5.22 (m, 1H) 5.17-4.94 (m, 2H), 3.95 (t, 2H, J=7.6), 2.39-2.29 (m, 2H), 2.17-1.99 (M, 2H), 1.99-1.85 (m, 2H), 1.85-1.72 (m, 2M), 1.68-1.55 (m, 2H); MS (m/z) 293.3 (MH+ 100%).

### Example 25

### Benzyl 4-[3-butyl-5-(diaminomethylene)-2,4,6-trioxotetrahydropyrimidin-1(2H)-yl]piperidine-1-carboxylate

### Preparation of intermediate benzyl 4-[3-butyl-5-(1,3-dithian-2-ylidene)-2,4,6-trioxotetrahydropyrimidin-1(2H)-yl]piperidine-1-carboxylate

To a solution of 4-amino-1-benzyl-piperidine (10.0 mmol) in 10 mL of dichloromethane at rt was added butyl isocyanate (10.0 mmol) in 5 mL of dichloromethane. The solution stirred for 1 h and then concentrated to a white solid. A THF solution of the residue was cooled to −10° C. under N₂ and Cbz-Cl (7.0 mmol) in 5 mL THF was added dropwise. The reaction was slowly warmed to rt with stirring for 3 h. Upon concentration the residue was subjected to chroatography on silica gel (100% EtOAc) and obtained 0.350 g of a white solid (30% yield). The urea was dissolved in Ac₂OH (0.5 mL) and Ac₂O(0.5 mL) and malonic acid (0.104 g, 1.0 mmol) was added. The mixture was heated to 80° C. for 1.5 h and then cooled and concentrated. The crude barbituric acid was dissolved in 1.0 mL of DMSO and then carbon disulfide (0.149 mL, 2.47 mmol) and triethylamine (0.458 mL, 3.29 mmol) were added. The reaction stirred for 90 min at rt and then 1,3 dibromopropane (0.083 mL, 0.82 mmol) was added. The solution stirred for another 6 h at rt and then diluted with EtOAc (10 mL) and water (3 mL). The organics were washed with brine and then dried (Na₂SO₄), filtered and concentrated. Chromatography on silica gel using 1:1 hexanes:EtOAc eluted intermediate benzyl 4-[3-butyl-5-(1,3-dithian-2-ylidene)-2,4,6-trioxotetrahydropyrimidin-1(2H)-yl]piperidine-1-carboxylate as a yellow solid (0.303 g, 71% yield) after drying under reduced pressure. Into a sealable pressure tube was added a suspension of 0.300 g of the above dithiane intermediate in 5 mL of 2.0M N in methanol solution. The tube was sealed and the contents stirred at 110° C. for 4 h. The solution was cooled and concentrated. Chromatography of the residue on silica gel eluting with 2:1 EtOAc:hexanes gave the title compound as a yellow oil (0.140 g, 54% yield): ¹H NMR (400 MHz, CDCl₃) δ 10.20 (bs, 2H), 7.38-7.27 (m, 5H), 5.98 (bs, 2H), 5.10 (s, 2H), 4.94, m, 1H), 4.27 (bm, 2H), 3.85 (t, 2H, J=7.6 Hz), 2.84 (bm, 2H), 2.65-2.56 (m, 2H), 1.60-1.53 (m, 4H), 1.40-1.30 (m, 2H), 0.93 (t, 3H, J=7.3 Hz).

### Example 26

### 4-(2λ⁵-Triaza-1,2-dienyl)benzyl 4-[3-butyl-5-(diaminomethylene)-2,4,6-trioxotetrahydropyrimidin-1(2H)-yl]piperidine-1-carboxylate

Benzyl 4-[3-butyl-5-(diaminomethylene)-2,4,6-trioxotetrahydropyrimidin-1(2H)-yl]piperidine-1-carboxylate (example 25, 0.130 g, 0.293 mmol) was dissolved in MeOH and 10% Pd/C was added. The reaction was subjected to 1 atm H₂ for 1 h, filtered over Celite and then resubjected to the reaction conditions. The reaction was complete within 20 min. to give a quantitative yield of intermediate amine. A DMF solution of the amine (0.045 g, 0.145 mmol), with triethylamine (0.020 mL) and 4-nitrophenyl 4-(2λ⁵-triaza-1,2-dienyl)benyl carbonate (J. Chem. Soc. PerkinTrans. I 1996, 11, 1205-1212) (0.062 g, 0.218 mmol) was stirred for 6 h at rt and then concentrated. The residue was redissolved in dichloromethane and the organics washed with 10% HCl, H₂O, followed by sat'd NaHCO₃, and brine. The organics were dried (Na₂SO₄), filtered and concentrated. Chromatography on silica gel eluting with 1:1 hexanes:EtOAc gave 0.055 g of the title compound as a white solid after drying under reduced pressure (79% yield): ¹H NMR (400 MHz, DMSO-d₆) δ 9.49 (bs, 2H), 7.39 (d, 2H, J=8.4 Hz), 7.30 (bs, 2H), 7.10 (d, 2H, J=8.5 Hz), 5.04 (s, 2H), 4.86 (m, 1H), 4.06-4.03 (bm, 2H), 3.71 (t, 2H, J=7.3 Hz), 2.94-2.74 (m, 2H), 2.42-2.32 (m, 2H), 1.48-1.39 (m, 4H), 1.27-1.18 (m, 2H), 0.85 (t, 3H, J=7.3 Hz); MS (m/z) 507 (M+Na).

### Example 27

### Benzyl 3-[3-butyl-5-(diaminomethylene)-2,4,6-trioxotetrahydropyrimidin-1(2H)-yl]pyrrolidine-1-carboxylate

### 27(a) Preparation of intermediate 2-(1-benzylpyrrolidin-3-yl)-1H-isoindole-1,3(2H)-dione

A solution of 1-benzylpyrrolidin-3-amine (5.73 mmol), N-carbethoxyphthalimide (5.73 mmol) and Et₃N (8.02 mmol) in THF (17.5 mL) was brought to reflux under N₂. After 3 h the solution was cooled to rt and concentrated in vacuo. The residue was partitioned between water and CH₂Cl₂, the organic layer was then washed with brine, dried over Na₂SO₄ and concentrated. The residue was purified by flash chromatography (75% EtOAc/hexanes→EtOAc), affording 4.31 mmol (75%) of intermediate 2-(1-benzylpyrrolidin-3-yl)-1H-isoindole-1,3(2H)-dione: ¹H NMR (300 MHz, CDCl₃) 7.77-7.87 (m, 2H), 7.65-7.75 (m, 2H), 7.19-7.40 (m, 5H), 4.91 (app. quint, 1H, J=8.1 Hz), 3.70 (s, 2H), 3.05 (t, 1H, J=8.6 Hz), 2.93-3.01 (m, 1H), 2.84 (q, 1H, J=8.6 Hz), 2.68 (t, 1H, J=8.6 Hz), 2.16-2.31 (m, 2H) ppm.

### 27(b) Preparation of intermediate benzyl 3-(1,3-dioxo-1,3-dihydro-2H-isoindol-2-yl)pyrrolidine-1-carboxylate

To a solution of 2-(1-benzylpyrrolidin-3-yl)-1H-isoindole-1,3(2H)-dione (4.18 mmol) in THF (8 mL) at −10° C. was added a solution of benzyl chloroformate (8.37 mmol) in THF (2 mL), dropwise over 3 min. The resulting mixture was allowed to warm to RT over 4 h, and concentrated in vacuo. The residue was purified by flash chromatography (25% EtOAc/hexanes), yielding 2.71 mmol (65%) of benzyl 3-(1,3-dioxo-1,3-dihydro-2H-isoindol-2-yl)pyrrolidine-1-carboxylate as a colorless glass: ¹H NMR (300 MHz, CDCl₃) 7.79-7.91 (m, 2H), 7.67-7.78 (m, 2H), 7.27-7.46 (m, 5H), 5.16 (s, 2H), 4.89 (app. quint, 1H, J=8.4 Hz), 2.64 (app. quint, 1H, J=9.7), 2.08-2.77 (m, 1H) ppm.

### 27(c) Preparation of benzyl 3-[3-butyl-5-(diaminomethylene)-2,4,6-trioxotetrahydropyrimidin-1(2H)-yl]pyrrolidine-1-carboxylate

To a mixture of benzyl 3-(1,3-dioxo-1,3-dihydro-2H-isoindol-2-yl)pyrrolidine-1-carboxylate (2.24 mmol) in i-PrOH/H₂O (6:1, 21 mL) at RT was added NaBH₄ (11.2 mmol), portionwise over 3 min. The resulting mixture was stirred 45 min and AcOH was added dropwise until an approximate pH of 5 was reached. The resulting mixture was then refluxed for 1 h, cooled to RT, poured into water, and the pH adjusted to 1 by addition of 10% (v/v) HCl solution. The aqueous mixture was extracted with Et₂O (×2) and adjusted to pH 10 by addition of satd Na₂CO₃ solution. The resulting mixture was extracted with EtOAc (×3). Combined organics were washed (H₂O, brine), dried (Na₂SO₄) and concentrated, affording 337 mg colorless oil which was used without further purification. The remaining synthetic steps were identical to those described in example 3. Thus, from the above oil was obtained 241 mg (87%) benzyl 3-(3-butyl-2,4,6-trioxotetrahydropyrimidin-1(2H)-yl)pyrrolidine-1-carboxylate as an orange gum, which in turn yielded 172 mg (55%) of intermediate benzyl 3-[3-butyl-5-(1,3-dithian-2-ylidene)-2,4,6-trioxotetrahydropyrimidin-1(2H)-yl]pyrrolidine-1-carboxylate. From the dithiane intermediate was obtained 74 mg (51%) of the title compound as a colorless foam. ¹H NMR (300 MHz, CDCl₃) 9.89 (br. s, 2H), 7.27-7.41 (m, 5H), 6.26 (br. s, 2H), 5.66 (app. quint, 1H, J=8.5 Hz), 5.05-5.20 (m, 2H, Ph-C₂), 3.75-3.92 (m, 4H), 3.57-3.75 (m, 1H), 3.39-3.54 (m, 1H), 2.45-2.67 (m, 1H), 2.05-2.20 (m, 1H), 1.49-1.66 (m, 2H), 1.36 (app. sext, 2H, J=7.5 Hz), 0.93 (t, 3H, J=7.3 Hz) ppm.

### Example 28

### 1-Butyl-5-(diaminomethylene)-3-(3,5-dimethylisoxazol-4-yl)pyrimidine-2,4,6(1H,3H,5H)-trione

Preparation of intermediate 1-butyl-3-(3,5-dimethylisoxazol-4-yl)-5-(1,3-dithian-2-ylidene)pyrimidine-2,4,6(1H, 3H, 5H)-trione: To a solution of butylamine (0.099 mL, 1.0 mmol) in 1.5 mL of dichloromethane at rt was added 3,5 dimethylisoxazol-4-yl isocyanate (0.138 g, 1.0 mmol) in 1 mL of dichloromethane. The solution stirred for 1 h and then concentrated to a white solid. The crude urea was dissolved in AcOH (0.5 mL) and Ac₂O (0.5 mL) and malonic acid (0.104 g, 1.0 mmol) was added. The mixture was heated to 80° C. for 1.5 h and then cooled and concentrated. The crude barbituric acid was dissolved in 1.0 mL of DMSO and then carbon disulfide (0.181 mL, 3.0 mmol) and triethylamine (0.557 mL, 4.0 mmol) were added. The reaction stirred for 90 min at rt and then 1,3 dibromopropane (0.101 mL, 1.0 mmol) was added. The solution was stirred for 6 h at rt and then diluted with EtOAc (10 mL) and water (3 mL). The organics were washed with brine and then dried (Na₂SO₄), filtered, and concentrated. Chromatography on silica gel eluting with 1:1 hexanes:EtOAc gave intermediate 1-butyl-3-(3,5-dimethylisoxazol-4-yl)-5-(1,3-dithian-2-ylidene)pyrimidine-2,4,6(1H, 3H, 5H)-trione as a yellow solid (0.230 g, 58% yield) after drying under reduced pressure.

Preparation of Title Compound:

Into a sealable pressure tube was added a suspension of 0.230 g of the above dithiane intermediate in 5 mL of 2.0M N, in methanol solution. The tube was sealed and the contents stirred at 110° C. for 4 h. The solution was cooled and concentrated. Chromatography of the residue on silica gel eluting with 4:1 EtOAc:hexanes gave the title compound as a white solid (0.100 g, 54% yield) upon drying in vacuo: ¹H NMR (400 MHz, CDCl₃) δ 10.2 (bs, 1H), 9.9 (bs, 1H), 5.6 (bs, 2H), 3.93 (t, 2H, J=7.4 Hz), 2.27 (s, 3H), 2.13 (s, 3H), 1.65-1.58 (m, 2H), 1.42-1.34 (m, 2H), 0.94 (t, 3H, J=7.3 Hz); MS (m/z) 322 (MH⁺).

### Example 29

### 6-Amino-1,3-dibutyl-2,4-dioxo-1,2,3,4-tetrahydropyrimidine-5-carbothioamide

29(a) Preparation of Intermediate 6-amino-1,3-dibutyl-N-(4-methoxybenzyl)-2,3-dioxo-1,2,3,4-tetrahydropyrimidine-5-carbothioamide: To 445 mg (1.86 mmol) of 6-amino-1,3-dibutyl-1H-pyrimidine-2,4-dione (J. Med. Chem. 1994, 37 (20) 3373-3382) in DMF (5 mL) was added 1.0 g (5.58 mmol) of 1-isothiocyanato-4-methoxybenzene at 23° C. The solution was heated to 100° C. for 16 hr. Upon cooling, ethyl acetate (30 mL) was added and the solution washed with three portions of water (20 mL) and brine (20 mL), dried over Na₂SO₄, concentrated then purified by silica gel chromatography eluting with hexanes/ethyl acetate solvent to give 540 mg (70%) of 6-amino-1,3-dibutyl-N-(4-methoxybenzyl)-2,3-dioxo-1,2,3,4-tetrahydropyrimidine-5-carbothioamide as a yellow oil: ¹H NMR (400 MHz, CDCl₃) δ 12.79 (s, 1H), 7.28 (d, 2H, f=7.6), 6.87 (d, 2H, J=7.6), 4.80 (d, 2H, J=4.8), 3.99-3.84 (m, 4H), 3.79 (s, 3H), 1.74-1.52 (m, 4H), 1.48-1.31 (m, 4H), 0.98 (t, 3H, J=7.2), 0.91 (t, 3H, J=7.2); MS (m/z) 419.4 (MH+ 100%).

### 29(b) Preparation of 6-amino-1,3-dibutyl-2,4-dioxo-1,2,3,4-tetrahydropyrimidine-5-carbothioamide

To 30% HBr in acetic acid (5 mL), was added 180 mg (0.43 mmol) of 6-amino-1,3-dibutyl-N-(4-methoxybenzyl)-2,3-dioxo-1,2,3,4-tetrahydropyrimidine-5-carbothioamide. The mixture was heated to 80° C. for 4 hr. Upon cooling, ethyl acetate (30 mL) was added and the solution washed with three portions of water (20 mL), sat. NaHCO₃ (20 mL) and brine (20 mL), dried over Na₂SO₄, concentrated then purified by silica gel chromatography eluting with hexanes/ethyl acetate solvent to give 62 mg (48% yield) of the title compound as a yellow solid: MS (m/z) 299.4 (MH+ 100%).

What is claimed is:

1. A compound of formula (I) 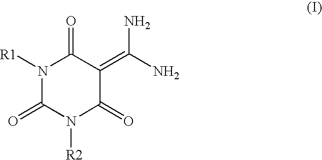
wherein,
R¹ and R² are the same or are different and are C₁₋₈ alkyl, C₂₋₈ alkylene, C₃₋₈ cycloalkyl, aryl, heteroaryl, heterocycloalkyl, C₃₋₆cycloalkylaryl, or heterocycloaryl; wherein said alkyl, alkylene, cycloalkyl, aryl, heteroaryl, heterocyclyl, cycloalkylaryl, or heterocycloaryl are unsubstituted or substituted by one or more groups selected from the group consisting of halogen, C₁₋₈ alkyl, C₁₋₈alkoxy, C₁₋₈thioalkoxy, cycloalkyl, aryl, heteroaryl, heterocycloalkyl, CF₃, SCF₃, NHC(O)ₙR⁵, S(O)ₘR⁵, S(O)₂NR⁵R⁶, C(S)NR⁵R⁶, CONR⁵R⁶, C(O)ₙR⁵;
n is 0, 1 or 2;
m is 0, 1 or 2;
R⁵ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃;
R⁶ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃, or when R¹ and/or R² contains S(O)₂NR⁵R⁶, CONR⁵R⁶, or C(S)NR⁵R⁶, then R⁵R⁶ together with the nitrogen may form a heterocyclic ring; or
a pharmaceutically acceptable salt or solvate thereof.

2. A compound of formula (II) 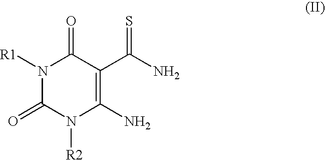
wherein,
R¹ and R² are the same or are different and are C₁₋₈ alkyl, C₂₋₈ alkylene, C₃₋₈ cycloalkyl, aryl, heteroaryl, heterocycloalkyl, C₃₋₆ cycloalkylaryl, or heterocycloaryl; wherein said alkyl, alkylene, cycloalkyl, aryl, heteroaryl, heterocyclyl, cycloalkylaryl, or heterocycloaryl are unsubstituted or substituted by one or more groups selected from the group consisting of halogen, C₁₋₈ alkyl, C₁₋₈alkoxy, C₁₋₈thioalkoxy, cycloalkyl, aryl, heteroaryl, heterocycloalkyl, CF₃, SCF₃, NHC(O)ₙR⁵, S(O)ₘR⁵, S(O)₂NR⁵R⁶, C(S)NR⁵R⁶, CONR⁵R⁶, C(O)ₙR⁵;
n is 0, 1 or 2;
m is 0, 1 or 2;
R⁵ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃;
R⁶ is hydrogen, alkyl, aryl, alkylaryl, heterocycloalkyl, or heteroaryl and is unsubstituted or substituted by one or more groups selected from the group consisting of alkyl, C₁₋₈alkoxy, aryl, heteroaryl, halogen, NO₂, CN, N₃, SCF₃, and CF₃, or when R¹ and/or R² contains S(O)₂NR⁵R⁶, CONR⁵R⁶, or C(S)NR⁵R⁶, then R⁵R⁶ together with the nitrogen may form a heterocyclic ring; or
a pharmaceutically acceptable salt or solvate thereof.

3. A compound of claim 1 wherein in formula (I) R¹, R² are the same or are different and are independently C₃₋₆ alkyl, C₃₋₆ alkylene, C₃₋₈cycloalkyl, C₄₋₆ alkylaryl, C₃₋₄cycloalkylaryl, heterocycloaryl or heterocycloalkyl. Said C₃₋₆alkyl or heterocycloalkyl may be optionally substituted with NHC(O)ₙR⁵ or C(O)ₙR⁵ wherein n is 2 and R⁵ is lower alkylaryl as herein defined wherein said lower alkylaryl may be optionally substituted with one or more groups selected from F, NO₂, or N₃.

4. A compound according to claim 3 wherein R² is n-butyl and R¹ is

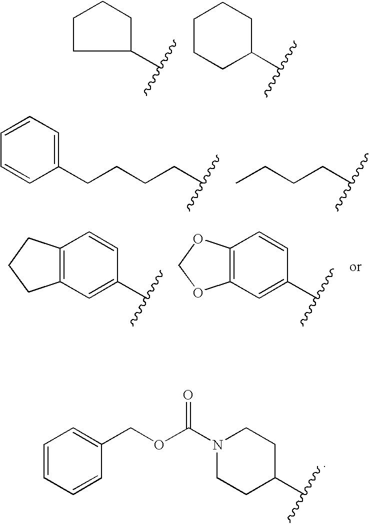

5. A compound according to claim 3 which is
1,3-dicyclohexyl-5-(diaminomethylene)pyrimidine-2,4,6(1H,3H,5H)-trione,
1-butyl-5-(diaminomethylene)-3-(2-methylbutyl)pyrimidine-2,4,6(1H,3H,5H)-trione,
1-butyl-5-(diaminomethylene)-3-(2,3-dihydro-1H-inden-2-yl)pyrimidine-2,4,6(1H,3H,5H)-trione,
1-butyl-5-(diaminomethylene)-3-{4-[(trifluoromethyl)thio]phenyl}pyrimidine-2,4,6(1H,3H,5H)-trione,
1-butyl-5-(diaminomethylene)-3-mesitylpyrimidine-2,4,6(1H,3H,5H)-trione,
1-butyl-5-(diaminomethylene)-3-(2,4-difluorophenyl)pyrimidine-2,4,6(1H,3H,5H)-trione,
1-butyl-5-(diaminomethylene)-3-(2-fluorophenyl)pyrimidine-2,4,6(1H,3H,5H)-trione,
1-butyl-3-(cyclohexylmethyl)-5-(diaminomethylene)pyrimidine-2,4,6(1H,3H,5H)-trione,
1-butyl-3-cycloheptyl-5-(diaminomethylene)pyrimidine-2,4,6(1H,3H,5H)-trione,
1-butyl-3-cyclooctyl-5-(diaminomethylene)pyrimidine-2,4,6(1H,3H,5H)-trione,
1-butyl-5-(diaminomethylene)-3-(3-phenylcyclopentyl)pyrimidine-2,4,6(1H,3H,5H)-trione,
1-butyl-5-(diaminomethylene)-3-(5-phenylpentyl)pyrimidine-2,4,6(1H,3H,5H)-trione,
1-[3-(benzyloxy)phenyl]-3-butyl-5-(diaminomethylene)pyrimidine-2,4,6(1H,3H,5H)-trione,
benzyl 3-[3-butyl-5-(diaminomethylene)-2,4,6-trioxotetrahydropyrimidin-1(2H)-yl]propylcarbamate,
4-nitrobenzyl 3-[3-butyl-5-(diaminomethylene)-2,4,6-trioxotetrahydropyrimidin-1(2H)-yl]propylcarbamate,
4-fluorobenzyl 3-[3-butyl-5-(diaminomethylene)-2,4,6-trioxotetrahydropyrimidin-1(2H)-yl]propylcarbamate,
4-(2λ⁵-triaza-1,2-dienyl)benzyl 3-[3-butyl-5-(diaminomethylene)-2,4,6-trioxotetrahydropyrimidin-1(2H)-yl]propylcarbamate,
1-but-3-enyl-3-cyclopentyl-5-(diaminomethylene)pyrimidine-2,4,6(1H,3H,5H)-trione,
4-(2λ⁵-triaza-1,2-dienyl)benzyl 4-[3-butyl-5-(diaminomethylene)-2,4,6-trioxotetrahydropyrimidin-1(2H)-yl]piperidine-1-carboxylate,
benzyl 3-[3-butyl-5-(diaminomethylene)-2,4,6-trioxotetrahydropyrimidin-1(2H)-yl]pyrrolidine-1-carboxylate,
1-butyl-5-(diaminomethylene)-3-(3,5-dimethylisoxazol-4-yl)pyrimidine-2,4,6(1H,3H,5H)-trione,
1,3-dibutyl-5-(diaminomethylene)pyrimidine-2,4,6(1H,3H,5H)-trione,
1-butyl-5-(diaminomethylene)-3-(4-phenylbutyl)pyrimidine-2,4,6(1H,3H,5H)-trione,
benzyl 4-[3-butyl-5-(diaminomethylene)-2,4,6-trioxotetrahydropyrimidin-1(2H)-yl]piperidine-1-carboxylate,
1-butyl-3-cyclopentyl-5-(diaminomethylene)pyrimidine-2,4,6(1H,3H,5H)-trione,
1-butyl-5-(diaminomethylene)-3-(2,3-dihydro-1H-inden-5-yl)pyrimidine-2,4,6(1H,3H,5H)-trione,
1-(1,3-benzodioxol-5-yl)-3-butyl-S-(diaminomethylene)pyrimidine-2,4,6(1H,3H,5H)-trione,
1-butyl-3-cyclohexyl-5-(diaminomethylene)pyrimidine-2,4,6(1H,3H,5H)-trione
1,3-dibutyl-5-(diaminomethylene)pyrimidine-2,4,6(1H,3H,5H)-trione,
1-butyl-5-(diaminomethylene)-3-(4-phenylbutyl)pyrimidine-2,4,6(1H,3H,5H)-trione,
benzyl 4-[3-butyl-5-(diaminomethylene)-2,4,6-trioxotetrahydropyrimidin-1(2H)-yl]piperidine-1-carboxylate,
1-butyl-3-cyclopentyl-5-(diaminomethylene)pyrimidine-2,4,6(1H,3H,5H)-trione,
1-butyl-5-(diaminomethylene)-3-(2,3-dihydro-1H-inden-5-yl)pyrimidine-2,4,6(1H,3H,5H)-trione,
1-(1,3-benzodioxol-5-yl)-3-butyl-5-(diaminomethylene)pyrimidine-2,4,6(1H,3H,5H)-trione,
1-butyl-3-cyclohexyl-5-(diaminomethylene)pyrimidine-2,4,6(1H,3H,5H)-trione, or
a pharmaceutically acceptable salt thereof.

6. A compound according to claim 2 wherein in formula (II) R¹, R² are the same or are different and are independently C₃₋₆ alkyl, C₃₋₆ alkylene, C₃₋₈cycloalkyl, C₄₋₆ alkylaryl, C₃₋₄cycloalkylaryl, heterocycloaryl or heterocycloalkyl. Said C₃₋₆ alkyl or heterocycloalkyl may be optionally substituted with NHC(O)ₙR⁵ or C(O)ₙR⁵ wherein n is 2 and R⁵ is lower alkylaryl as herein defined wherein said lower alkylaryl may be optionally substituted with one or more groups selected from F, NO₂, or N₃.

7. A compound according to claim 6 wherein R² is n-butyl and R¹ is

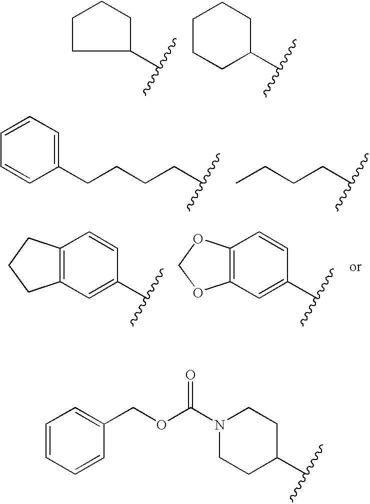

8. A compound according to claim 6 which is
6-amino-1,3-dibutyl-2,4-dioxo-1,2,3,4-tetrahydropyrimidine-5-carbothioamide or a pharmaceutically acceptable salt thereof.

9. A pharmaceutical composition comprising a compound of claim 1 in admixture with a pharmaceutically excipient.

10. A pharmaceutical composition comprising a compound of claim 2 in admixture with a pharmaceutically excipient.

11. A method for the prophylaxis of or treating osteoporosis in a mammal comprising administering a effective amount of a compound of claim 1 alone or in the form of a pharmaceutically acceptable excipient.

12. A method for the prophylaxis of or treating osteoporosis in a mammal comprising administering a effective amount of a compound of claim 2 alone or in the form of a pharmaceutically acceptable excipient.
